(19) United States
(12) Patent Application Publication
(10) Pub. No.: US 2007/0202495 A1
(43) Pub. Date: Aug. 30, 2007

(54) USE OF RESISTIVE-PULSE SENSING WITH SUBMICROMETER PORES OR NANOPORES FOR THE DETECTION OF THE ASSEMBLY OF SUBMICROMETER OR NANOMETER SIZED OBJECTS

(76) Inventors: Michael Mayer, Ann Arbor, MI (US); Jeffrey D. Uram, Youngstown, MI (US); Kevin Ke, Temple City, MI (US); Alan J. Hunt, Plymouth, MI (US)

Correspondence Address: HARNESS, DICKEY & PIERCE, P.L.C., P.O. BOX 828, BLOOMFIELD HILLS, MI, 48303, US

(21) Appl. No.: 11/671,171

(22) Filed: Feb. 5, 2007

Related U.S. Application Data
(60) Provisional application No. 60/765,758, filed on Feb. 6, 2006.

(51) Int. Cl. C12Q 1/70; C12Q 1/68; G01N 33/53; G01N 33/554; G01N 33/569

(52) U.S. Cl. 435/5; 435/6; 435/7.1; 435/7.32; 977/902; 977/924

(57) ABSTRACT

Methods and compositions for detecting the assembly of complexes include providing a solution where a first portion is separated from a second portion via a submicrometer pore, submicrometer tube or channel, nanopore, or nanotube or channel. One or more submicrometer or nanometer sized object(s) is added to the first portion of the solution. Due to molecular interactions, these objects assemble to form complexes consisting of two or more submicrometer or nanometer sized objects. Passage of a complex from the first portion of the solution through the submicrometer pore, submicrometer tube or channel, nanopore, or nanotube or channel to the second portion of the solution is detected using resistive pulse sensing. This sensing methodology may comprise detecting formation of complexes in real-time and/or may comprise detecting preassembled complexes.

### CROSS-REFERENCE TO RELATED APPLICATIONS

This application claims the benefit of U.S. Provisional Application No. 60/765,758, filed on Feb. 6, 2006. The disclosure of the above application is incorporated herein by reference.

### INTRODUCTION

The present disclosure relates to methods for detecting the solution-based assembly of complexes from submicrometer or nanometer sized objects, including the active assembly of complexes and/or preassembled complexes, using a submicrometer pore, submicrometer tube or channel, nanopore, nanotube or channel, and the resistive-pulse technique.

The interaction of submicrometer and nanometer sized objects in solution and the formation of complexes between two or more submicrometer or nanometer size objects in solution is important in many nanotechnological, biological, and chemical processes and compositions. For example, many diagnostics, such as immunoassays, are designed to detect binding events and formation of various complexes of one or more objects or particles. Complexes of submicrometer and nanometer sized objects of interest may include self-assembling complexes and/or assembly of complexes comprising different objects. Complexes may also include coupling between monovalent objects or complexes of polyvalent objects, including several objects to even thousands of objects or more. For example, assembly of complexes of submicrometer or nanometer sized objects may include complexes formed by protein-protein interactions, protein-virus interactions, nanoparticle-protein interactions, nanoparticle-virus interactions, nanoparticle-nanoparticle interactions, nanoparticle-template interactions, binding of monoclonal or polyclonal antibodies to antigens, binding of monoclonal or polyclonal antibodies to antigens immobilized on objects, polynucleotide-polynucleotide interactions, and protein-polynucleotide interactions, to name a few.

There are several methods available to detect the assembly of submicrometer or nanometer sized objects into complexes. These methods include scanning electron microscopy (SEM), transmission electron microscopy (TEM), atomic force microscopy (AFM), and light scattering techniques (e.g. dynamic light scattering). In order to perform SEM or TEM, the assembly needs to be dried out and placed in a high vacuum environment. Either of these steps can modify the nature of the assembly so that the true solution-based nature of the complex is not revealed. SEM, TEM, and AFM are very slow measurement techniques which require hours of work to produce measurements on tens of assemblies. Furthermore, such techniques require skilled operators and equipment costing >$50,000. While light scattering techniques can be performed quickly on solution-based samples, they have difficulty characterizing polydisperse samples since they do not measure individual objects in solution (inaccurate results are produced); polydisperse samples are commonly formed during the assembly process.

Other techniques for characterizing the formation of complex assemblies in solution make use of labeled antibodies. Detection labels, including radioisotopes, chemiluminescent conjugates, or calorimetric assays, may present handling issues, short lifespans, and may alter the structure and/or binding characteristics of the object of interest depending on the location and/or nature of the conjugation. Moreover, some of these methods use indirect measurements of complex assembly, for example, by using secondary antibodies and/or label or detection affinities independent of the binding event of the objects of interest.

Accordingly, there is a need for improved methods and compositions for detecting the solution-based assembly of submicrometer and nanometer sized objects. A method that is non-destructive and does not require immobilization or modification of the object would be advantageous. As such, methods for examining submicrometer or nanometer sized complexes in their native state would further preserve material for reuse or further analysis. The technique discussed here is non-destructive, does not require but can function with immobilization, examines each complex individually in solution, and can measure hundreds of complexes in a matter of minutes.

### SUMMARY

The present technology provides a method for detecting the assembly of complexes. In some embodiments, the method comprises providing a solution where a first portion is separated from a second portion via a single submicrometer pore, a single submicrometer channel or tube, a single nanopore, or a single nanochannel or tube. As used herein, a submicrometer pore can also be a submicrometer channel or submicrometer tube and a nanopore can also be a nanochannel or nanotube. One or more submicrometer or nanometer sized object(s) is added to the first portion of the solution. Due to molecular interactions, these objects assemble to form complexes consisting of two or more submicrometer or nanometer sized objects. Passage of a complex from the first portion of the solution through the submicrometer pore or nanopore to the second portion of the solution is detected using resistive-pulse sensing. In some embodiments, complexes comprising different numbers of submicrometer or nanometer sized objects are detected. In some embodiments, a complex includes at least two submicrometer or nanometer sized objects, and in some embodiments, the at least two submicrometer or nanometer sized objects may be the same object or may be different objects.

In some embodiments, the method uses resistive-pulse sensing to detect a change in current, wherein the change is proportional to the volume of the complex. In some embodiments, the method uses resistive-pulse sensing to detect a number of resistive-pulses per time interval, wherein the number is correlated to the concentration of the complex. And in some embodiments, the method uses resistive-pulse sensing to detect a residence time of the complex in the submicrometer pore or nanopore, wherein the residence time is correlated to the velocity of the complex. Some embodiments also include a method that uses resistive-pulse sensing to detect blockage of the submicrometer pore or nanopore by the complex. In some embodiments, the complex may comprise detecting formation of complexes in real-time and/or may comprise detecting preassembled complexes.

The present technology also includes a method for identifying intermolecular interactions. In some embodiments, the method includes partitioning an electrolyte volume with a submicrometer pore or nanopore and establishing a concentration gradient of a submicrometer object across the submicrometer pore. A change in electrical signal is measured when a complex comprising at least two submicrometer objects traverses the submicrometer pore.

Some embodiments include measuring a change in electrical signal when a complex comprising at least two submicrometer or nanometer sized objects traverses the submicrometer pore. In some embodiments the measuring may further include determining the volume of the complex based on the change in current, determining the concentration of complex based on the number of resistive pulses per time interval, and/or determining the velocity of the complex based on the residence time of the complex in the submicrometer pore or nanopore.

Aspects of the present technology include a method that uses a submicrometer pore or nanopore to detect and characterize immune complexes consisting of proteins, such as staphylococcal enterotoxin B (an agent with bioterrorism potential) and polyclonal antibodies. Other aspects include methods for detecting and characterizing complexes assembled from submicrometer particles or nanoparticles. Further aspects include methods that use a submicrometer pore-based resistive-pulse sensor to 1) detect a specific virus or a virus specific antibody in solution, 2) probe the ability of an antibody to immunoprecipitate the virus, 3) determine the number of antibodies bound to individual virus particles, and 4) monitor the assembly of nanoparticles onto templates (e.g., antibodies onto viruses) in situ. Still further aspects include methods that use resistive-pulse sensing to estimate the affinity constant of a biological or synthetic interaction between two submicrometer or nanometer sized objects, for example such as for an antibody binding to its antigen. Other aspects include estimating the number of one submicrometer or nanometer sized object bound to another submicrometer or nanometer sized object in a complex. Further aspects include methods for detecting and determining the solubility of submicrometer or nanometer sized objects such as drug molecules or proteins and probing the crystallinity of the complexes that form.

The present technology affords several benefits including: the ability to detect the assembly of the complexes in real-time and/or to detect preassembled complexes; detection of complexes formed from objects in their native state; detection, characterization, and quantification of the complexes, such as an examination of the binding of antibodies to viruses; and the ability to estimate and/or determine the affinity constants of the interaction of two objects. Moreover, the present methods are rapid, label free, may require no immobilization or modification of the object of interest, and may achieve single-complex sensitivity by monitoring changes in electrical resistance when the complexes pass through the submicrometer pore or nanopore. In the case of biological samples, the complex of interest may be detected in complicated media such as serum. Furthermore, owing to the small equipment footprint of the present technology, submicrometer pore- or nanopore-based sensing of complexes may enable portable or high-throughput immunoassays for diagnostics and biodefense.

Further areas of applicability of the present teachings will become apparent from the detailed description provided herein. It should be understood that the detailed description and specific examples, while indicating some embodiments of the teachings, are intended for purposes of illustration only and are not intended to limit the scope of the teachings.

### DRAWINGS

The present teachings will become more fully understood from the detailed description and the accompanying drawings, wherein:

FIG. 1 illustrates laser-based fabrication of submicrometer pores with conical geometry;

FIG. 2 ilustrates a side a view of a resistive-pulse sensing setup according to one embodiment of the present teachings;

FIG. 3 illustrates time courses of the formation of immune complexes in solution;

FIG. 4 illustrates detection of staphylococcal enterotoxin B (SEB) by sensing the formation of immune complexes in media containing a complex sample matrix;

FIG. 5 illustrates a time course of the current peak amplitudes and volumes of immune complexes;

FIG. 6 illustrates a resistive-pulse sensing technique for detecting and characterizing the binding of antibodies to virus particles according to one embodiment of the present teachings;

FIG. 7 illustrates detection of an antibody-virus interaction using a submicrometer pore;

FIG. 8 illustrates kinetics of antibody binding at different rations of antibody to virus concentration and estimation of the maximum number of antibodies that can bind to the virus;

FIG. 9 illustrates the number of monoclonal anti-streptavidin antibodies from mouse bound per streptavidin-functionalized colloid, r, vs. the initial concentration of antibody in solution;

FIG. 10 illustrates a plot of the number of antibodies bound per colloid at equilibrium, r, as a function of the free antibody concentration at equilibrium;

FIG. 11 illustrates nanopore-based detection of protein aggregates and crystals according to one embodiment of the present teachings;

FIG. 12 illustrates SEM images of conical pores with diameters of 575 nm and 900 nm and determination of the relationship between peak amplitude and particle volume in conical pores with submicrometer diameter;

FIG. 13 illustrates histograms of the halfwidths of events caused by immune complexes and nanoparticles passing through submicrometer pores with conical geometry;

FIG. 14 illustrates power spectra of original current traces with and without events;

FIG. 15 illustrates the effect of the cutoff frequency used for low-pass filtering on the peak amplitudes of current events during passage of immune complexes through a submicrometer pore;

FIG. 16 illustrates microscope images to verify the specific formation of immune complexes;

FIG. 17 illustrates blockage of the submicrometer pore with a diameter of 650 nm by large immune complexes;

FIG. 18 illustrates time courses of the formation of immune complexes in a solution containing serum;

FIG. 19 illustrates a schematic design of a conical pore and the recording setup according to one embodiment of the present teachings;

FIG. 20 illustrates a plot of the average peak amplitude of the resistive-pulses caused by particles with a diameter of 100, 130, and 160 nm passing through a pore with a diameter of 575 nm versus particle volume;

FIG. 21 illustrates the determination of the bandwidth available during Coulter counting and the bandwidth required to resolve events;

FIG. 22 illustrates the effect of the cutoff frequency used for low-pass filtering on the peak amplitudes of current events during passage of viruses through a submicrometer pore;

FIG. 23 illustrates a close-up view of a single event due to the passage of a virus through the pore before and after decimation of data;

FIG. 24 illustrates a histogram of the half-widths of events due to the passage of viruses at different bandwidths in the absence and presence of antiserum to demonstrate that the bandwidth and data decimation did not distort the recorded signals;

FIG. 25 illustrates a histogram of the peak amplitudes of 1395 events caused by PBCV-1 without antibody bound passing through the pore as shown in FIG. 19; and illustrates the frequency of events versus the concentration of virus,

FIG. 26 illustrates microscopic observation of antiserum, control serum, and of virus antibody complexes; and

FIG. 27 illustrates a TEM image with individual measurements of the distance between virus particles in an aggregate.

### DESCRIPTION

The description of the following technology is merely exemplary in nature of the subject matter, manufacture, and use of the teaching disclosed herein, and is not intended to limit the scope, application, or uses of any specific invention claimed in this application, or in such other applications as may be filed claiming priority to this application, or patents issuing therefrom.

The present technology may rapidly detect the assembly of complexes formed of submicrometer or nanometer sized objects, with or without immobilization or labeling of the object, by combining a submicrometer pore or nanopore with resistive-pulse sensing to monitor the formation of complexes in solution. A “submicrometer pore,” as used herein, includes a submicrometer tube or channel, a nanopore, and nanotube or channel. In addition, a “submicrometer object,” as used herein, includes a nanometer sized object. Resistive-pulse sensing, also known as Coulter counting, monitors the transient change in resistance (resistive-pulse) that occurs when a particle passes through a submicrometer pore filled with electrolyte. As used herein, “resistive-pulse sensing” and “Coulter counting” are used synonymously, and may employ a Coulter counter device, which is also referred to simply as a Coulter counter.

Resistive-pulse sensing is used for detecting and analyzing microscale, and increasingly, nanoscale objects. As the sensitivity of a Coulter counter increases with decreasing pore diameter and length, various techniques may be used for the fabrication of membranes that contain a single submicrometer pore or nanopore. Pore-forming proteins in planar lipid bilayers (PLBs) may be used as nanopore sensors Fabricated structures, in comparison, can offer a high degree of robustness and withstand environmental stress such as vibration, pressure, extreme pH, and elevated temperatures. Fabricated nanopores and nanotubes may be used for resistive-pulse sensing to detect viruses, aggregation of colloids, DNA, nanoparticles, and proteins.

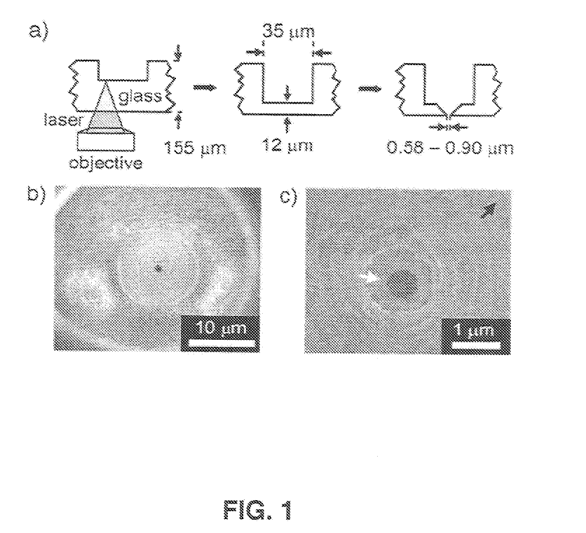

The present technology includes a nanomachining technique that employs femtosecond-pulsed lasers to fabricate submicrometer pore and nanopore structures in borosilicate glass coverslides. See FIG. 1 and Example 1 for an exemplary method of laser nanomachining. This technique has the advantage that it does not require masks, etching, or high vacuum and that it can fabricate in glass. Glass is an excellent substrate material owing to its low-noise properties, its chemical and mechanical robustness, and its amenability to surface functionalization. Furthermore, laser nanomachining is able to fabricate complicated 3D structures in optically transparent substrates. This enables generation of pores with a conical geometry and diameters of 575, 650 (shown in FIG. 1b, c), and 900 nm. See Example 2 for exemplary scanning electron micrograph (SEM) images of the 575 and 900 nm pores. The conical shape may produce low-resistance pores in thick (>1 mm) membranes that have low electrical capacitance. Decreasing the resistance increases the amplitude of resistive pulses as well as the rate of transport through the pore for a given pore diameter. Lowering the capacitance can reduce electrical-current noise, which permits recording at high bandwidths and increases the sensitivity of the Coulter counter.

An example of laser-based fabrication of submicrometer pores with conical geometry is shown in FIG. 1. The panels show: a) femtosecond-pulsed lasers enabled nanomachining of conical pores in glass with diameters as small as 575 nm; b) scanning electron microscope (SEM) image looking into the 35-mm cylinder of a pore (see a); c) SEM image focused on the narrowest part of the pore (diameter: 650 nm). The conical shape of the pores was confirmed by observing different focal planes with SEM (white arrow: in focus, black arrow: out of focus).

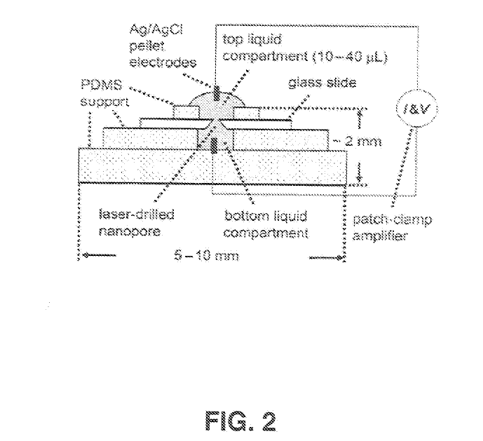

The glass slide with the pores may be mounted onto a fluidic setup, as shown in FIG. 2, made of poly(dimethylsiloxane) (PDMS) to characterize their electrical properties and to perform affinity assays. An exemplary composition for the recording buffer, resulting electrical resistances, and noise values of the pores are disclosed in Example 3.

The response of the nano-Coulter counter may be characterized by using synthetic nanoparticles. In a cylindrical pore, the resistive pulse from a spherical particle is proportional to the volume of the particle (as long as the particle diameter is less than ≈0.4 of the diameter of the pore). When objects or particles with diameters of 100, 130, and 160 nm are passed through a conical pore, a linear relationship is observed between the amplitude of the current peak and the particle volume, as disclosed in Example 2. This linear relationship, in conjunction with evidence that particles of the same volume, but varying shape, give rise to resistive pulses with similar amplitudes, makes it possible to estimate the volume of the immune complexes and consequently the number of objects (e.g., proteins) in a complex. An exemplary method is disclosed in Example 4.

FIG. 2 shows a cross-section view of an embodiment of the experimental setup constructed in accordance with the present teachings. A patch-clamp amplifier applies a constant voltage and detects small changes in current (pA-range) with fast time resolution (10-50 kHz). A poly(dimethylsiloxane) (PDMS) fluidic setup allows for replacement of solution on either side of the submicrometer pore.

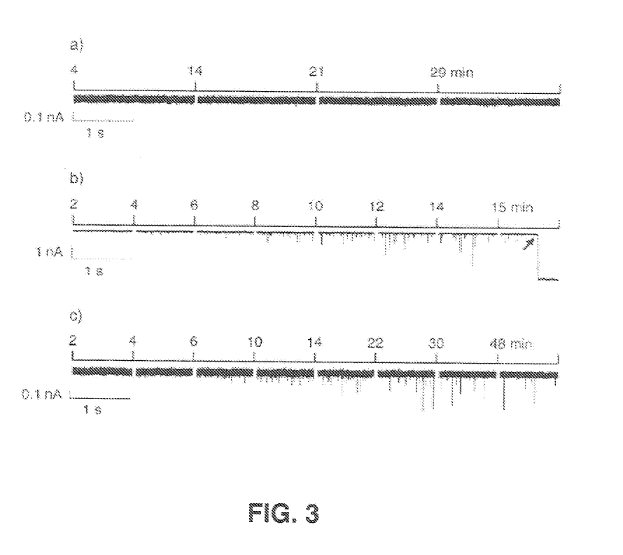

Resistive pulses that occur when complexes pass through the pore are monitored to detect and characterize the complexes. The exemplary antibody-antigen system investigated herein uses a goat anti-mouse antibody and a mouse monoclonal anti-baculovirus antibody as the antigen. To support the formation of complexes, Example 5 discloses experiments confirming immune complexes by phase contrast and fluorescence microscopy. Three different equimolar concentrations (15, 30, and 151 nm) of the antigen and the anti-mouse antibody were examined using a pore with a diameter of 650 nm. The assay can detect immune complexes at a concentration of 151 nM and 30 nM as shown by the resistive pulses in FIG. 3b, c; no immune complexes at a concentration of 15 nM of antibody and antigen were detected.

FIG. 3b, c shows that the amplitudes of many resistive pulses, caused by the immune complexes formed at a concentration of 151 nM, were considerably larger than those formed at 30 nM. This result indicates that the immune complexes grew larger at 151 nM than they did at 30 nM, and may explain why no immune complexes could be detected at a concentration of 15 nM. Indeed, the immune complexes that formed at a concentration of 151 nM grew so large that they eventually blocked the pore. In FIG. 3b, the arrow indicates the onset of pore blockage; see also Example 6.

FIG. 3 shows time courses of the formation of immune complexes in solution. The panels show: a) Control experiment with the antigen (mouse monoclonal anti-baculovirus antibody) and a nonspecific anti-rabbit antibody, both at a final concentration of 151 nM; b) At a final concentration of 151 nM of antigen and the specific anti-mouse antibody, detectable immune complexes formed rapidly and eventually blocked the pore (arrow). Note the y-scale of b) is ten times larger than the scale of the other traces owing to the large size of the immune complexes; c) At a lower antibody-antigen concentration (30 nM)₇ detectable immune complexes formed but were smaller and did not block the pore. Each current trace is composed of multiple, short-duration recordings (length 1-2 s; see marked scale) that were taken from data files recorded during the course of the experiment; a small gap separates each of these short recordings. The time in minutes, after the addition of anti-mouse or anti-rabbit antibody to the recording buffer that contained the antigen, is indicated above the beginning of each short recording. These recordings therefore represent short “snapshots” of the current activity throughout the entire experiment of several minutes duration. A pore with a diameter of 650 nm (FIG. 1b, c) was used for these experiments.

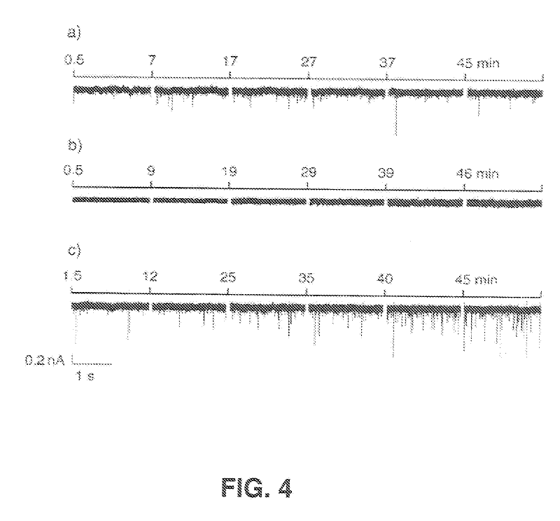

A control experiment was performed by using the same antigen and a nonspecific goat anti-rabbit antibody at a concentration of 151 nM. No immune complexes or pore blockage were detected in the presence of this control antibody, as shown in FIG. 3a. Subsequent addition of the anti-mouse antibody at a concentration of 151 nM produced detectable immune complexes within 3 minutes, and blockage of the pore, owing to large immune complexes (“immunospecific” blockage), occurred after approximately 9 minutes. This blockage provided a dramatic response (significant and permanent change in the resistance of the submicrometer pore) that could be sensed by using simple electronics with low time resolution. It may potentially be useful for disposable, ultrasmall, and portable low-power sensors for the detection of biowarfare agents such as staphylococcal enterotoxin B (SEB) (see FIG. 4), botolinum toxin, or ricin.

FIG. 4 shows the detection of staphylococcal enterotoxin B (SEB) by sensing the formation of immune complexes in media containing a complex sample matrix. The panels show: a) Current traces of anti-SEB serum only: one microliter of anti-SEB serum was added to 29 mL of recording buffer (the nonspecific events were caused by serum components that were not removed by a membrane filter with pores of 0.1 mm); b) Current traces of SEB only (final concentration: 200 nM); c) Current traces of SEB and anti-SEB serum: SEB at a final concentration of 200 nM and 1 mL of anti-SEB serum in a total volume of 32 mL. The addition of anti-SEB serum caused a significant increase in the number and size of events compared to a) above. Each current trace is composed of multiple, short duration (length 2 s, see marked scale) recordings that were taken from data files recorded at different times during the experiment; a small gap separates each recording. The time in minutes after the addition of anti-SEB serum, or SEB, to the recording buffer is indicated above the beginning of each short recording.

To test the ability of the pore-based sensor to detect proteins on a relevant system in complex media, staphylococcal enterotoxin B (SEB) was detected using sheep anti-SEB serum. SEB is a causative agent of food poisoning and has the potential for bioterrorism according to the National Institute of Allergy and Infectious Disease of the USA. The addition of the anti-SEB serum to a solution containing SEB caused a large increase in the size and number of detectable aggregates (i.e., complexes) when compared with the anti-SEB serum alone, as shown in FIG. 4. Similar results were obtained with a second system that employed rabbit antiserum to detect a monoclonal antibody, as disclosed in Example 7. These results demonstrate that submicrometer pore-based sensors can detect immune complexes in media that contain complex samples such as blood serum.

In addition to detecting immune complexes, and hence antigens or antibodies, submicrometer pore- or nanopore-based Coulter counting offers the possibility to evaluate specific properties of these complexes, such as their volume and growth rate. These properties are important as the size of an immune complex influences its physiological properties, for instance its clearance from circulation and its adherence to phagocytes. Studying polydisperse immune complexes is difficult owing to their large heterogeneity. Light-scattering techniques have been used; however, as they measure multiple particles at once, these techniques can be problematic for characterizing polydisperse samples. In contrast, Coulter counting measures each particle individually and therefore can provide information on the volume, polydispersity, and growth of the immune complexes with single-aggregate (i.e., single-complex) sensitivity.

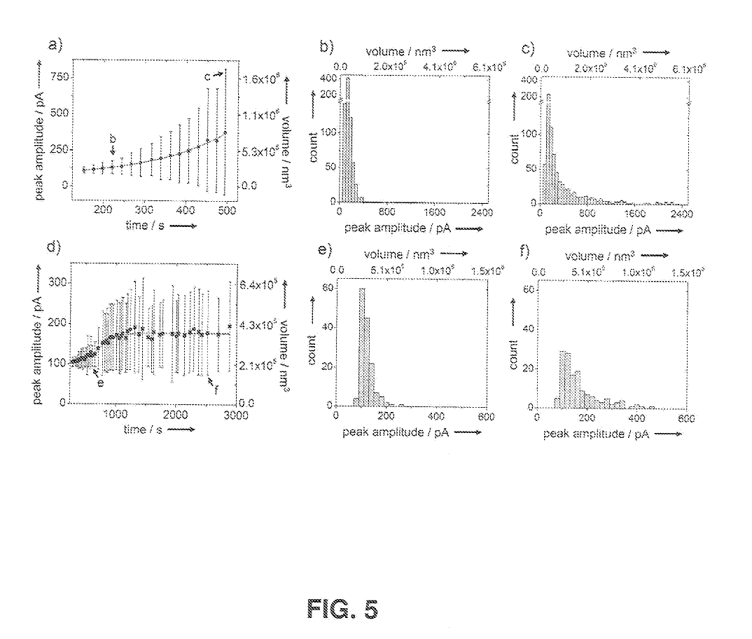

To demonstrate these capabilities, the increase in volume of immune complexes was monitored over time, as depicted in FIG. 5a, d. The general trend of the average peak amplitudes compares well with data obtained by light scattering. The sigmoidal shape in FIG. 5d may be a consequence of a thermodynamically stable size of the immune complexes. FIG. 5a, d shows that the standard deviation in the amplitude of the current peaks increased significantly during the growth of the immune complexes, therefore indicating a strong increase in the polydispersity of the complexes. Interestingly, the majority of the immune complexes sensed shortly after the addition of antibody (FIG. 5b, e) had volumes that were comparable to complexes that were sensed after 8 min (FIG. 5c) and 40 min (FIG. 5f). With increasing time, however, a fraction of the complexes reached volumes that were approximately two-times larger than that of the majority of the volumes (FIG. 5 f). This result suggests that the later stage of growth may have been caused by collisions between slowly diffusing complexes and may explain the relatively rare formation of complexes that are significantly larger than the majority.

FIG. 5 shows the time course of the current peak amplitudes and volumes of immune complexes. The panels show: a) Growth of immune complexes at a concentration of 151 nM of both antigen (mouse monoclonal anti-baculovirus antibody) and anti-mouse antibody. A first-order exponential function was fitted to the data. The small letters in graph a) corresponds to the time points from which the histograms shown in b) and c) were extracted; b) Peak amplitudes and volumes recorded at 240 s after the addition of anti-mouse antibody; c) Peak amplitudes and volumes recorded 490 s after addition of anti-mouse antibody. Note that 74% of the complexes maintained their volumes compared to b); however, a small fraction of complexes reached volumes that were up to ten times larger than in b); d) Growth of immune complexes at a concentration of 30 nM. A sigmoidal function was fitted to the data. The small letters in graph d) correspond to the time points from which the histograms shown in e) and f) were extracted. e) Peak amplitudes and volumes recorded at 610 s after the addition of anti-mouse antibody; f) Peak amplitudes and volumes recorded 2400 s after the addition of anti-mouse antibody. Note the occurrence of peak amplitudes with approximately two, three, and four times the change in current (≈200, 300, 400 pA) of those shown in e). Each point in a), d) reflects the average amplitude and aggregate volume obtained from peaks over a period of 20 s.

As a result of the linear relationship between the peak amplitude and volume of immune complexes, the number of proteins in an aggregate may be estimated by assuming a molecular volume of 347 nm³ for an immunoglobulin G antibody. The volumes of the immune complexes sensed by the pore with a diameter of 650 nm and an antibody antigen concentration of 151 nM ranged from 2.1×10⁵ to 6.0×10⁶ nm³, which corresponds to aggregates of 610 to 17,300 proteins.

Submicrometer pore-based detection of immune complexes using the present technology offers a general, rapid, label-free, and solution-based method for the detection of any submicrometer object, protein, or particle that can be triggered to form a detectable assembly, while providing information on the volume, growth, and polydispersity of individual aggregates or complexes. The detection limit of 30 nM for antigens compares favorably to other label-free detection techniques such as affinity capillary electrophoresis (ACE), gel-based immunoprecipitation, and direct immunoaggregation assays based on light scattering, all of which have detection limits between 10 and 1000 nM depending on the technique. Increasing the sensitivity of the Coulter counter (e.g. by reducing the diameter of the pore or the length of the pore) will increase the senstivity of the measurement thereby allowing detection of proteins at even lower concentrations (by allowing the detection of complexes that contain even fewer proteins). In addition to its benefits for affinity assays with small footprints and reagent requirements, the technology presented herein may be particularly useful for in situ, quantitative monitoring of controlled assemblies of nanoparticles (e.g., monitoring the number of nanoparticles in a complex, the speed of the formation of the complexes, etc.), thereby addressing an urgent need in nanotechnology.

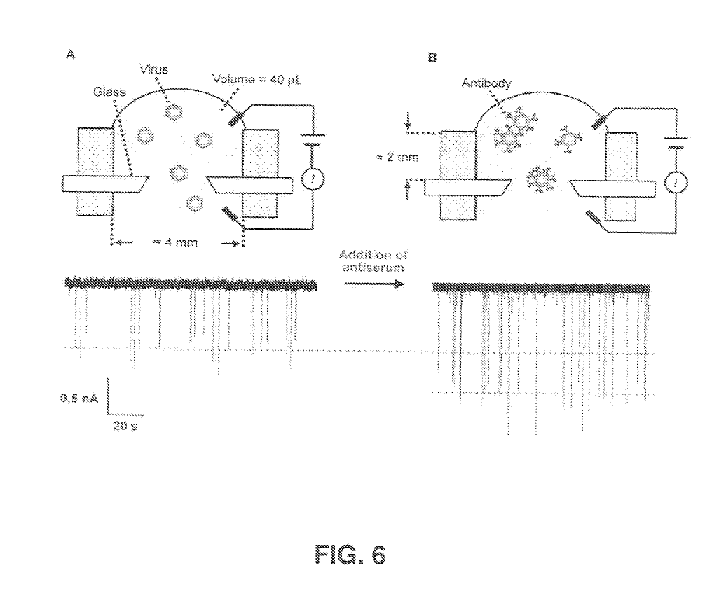

The present technology also includes the use of resistive-pulse sensing for the detection, characterization, and quantification of the binding of antibodies to intact virus particles. The technology includes a nondestructive method for detecting virus-specific antibodies in solution and for determining the number of antibodies bound to an intact virus in a physiological buffer. This label-free technique is able to operate with virus concentrations as low as 5×10⁷ particles/mL and establishes whether or not the antibody can aggregate (i.e., immunoprecipitate) the virus by forming a complex. As illustrated in FIG. 6, the approach uses laser-fabricated pores in glass and measures transient changes in current (so-called “resistive pulses”) by using Coulter counting experiments. In the present experiments, the reaction volume was 40 μL, but this value could be reduced to <10 μL via the integration of microfluidics. Due to the small size of the pores, this approach could potentially be miniaturized and performed in parallel for high-throughput applications.

In order to measure the resistive pulses caused by the passage of virus particles through the pore, a similar setup to FIG. 1 was used. It consisted of a patch-clamp amplifier with two Ag/AgCl electrodes and a conical pore with a diameter of 650 nm mounted in a poly-(dimethylsiloxane) (PDMS) fluidic setup The setup is disclosed in Example 8. The pore was fabricated in a borosilicate cover glass using a femtosecond-pulsed laser. Glass was chosen as the substrate because it is an excellent material for low-noise electrical recordings (i.e., low capacitance, low dielectric loss), and the conical shape of the pore provided enhanced sensitivity compared to cylindrical pores. Replacement of the solutions on either side of the pore was straightforward due to the fluidic setup, and the transparency of the entire assembly made it possible to observe the pore with a microscope when necessary.

Before examining the interaction of antibodies with virus particles, the response of the submicrometer pore to spherical nanoparticles of defined size and shape was characterized, as further disclosed in Example 9. A spherical particle passing through a cylindrical nanopore creates a resistive pulse with a peak amplitude proportional to the volume of the particle (as long as the particle diameter was less than ≈40% of the diameter of the pore). A linear relationship exists for spherical particles passing through conical pores, as shown in Example 2. The proportionality between current peak amplitude and particle volume for the conical pore was 3.9×10⁻⁴ pA/nm³. Virus particles are typically not perfectly spherical; however, experimental evidence suggests that the shape of particles that resemble spheroids does not influence the linear relationship between particle volume and peak amplitude. This linear correlation was used to estimate the change in the volume of PBCV-1 virus particles before and after antibody binding, shown in FIG. 6.

FIG. 6 illustrates the resistive-pulse technique for detecting and characterizing the binding of antibodies to virus particles. The panels show: A) Detection of virus particles before addition of antibodies: Single virions passing through the laser-fabricated conical pore cause a transient reduction in current (resistive pulse) as shown by the spikes (events) in the current trace. The dotted line represents the mean of a Gaussian curve fit to the distribution of the peak amplitudes of the events. The concentration of the virus was 4×10⁷ particles/mL and the average current passing through the pore for all experiments was ≈140 nA. B) Detection of virus particles after addition of antibodies: Binding of antibodies to the virus increases the volume of the particle leading to an increase in the peak amplitude when the viruses pass through the pore. The current trace displays events that were recorded 10-15 min after addition of the antiserum, which was at a final dilution of 0.001× the original antiserum. If the antibody is capable of causing aggregation of viruses, this approach makes it possible to identify dimers (and larger complexes of virus particles) by detecting events with approximately twice (three times, etc.) the peak amplitude of individual viruses.

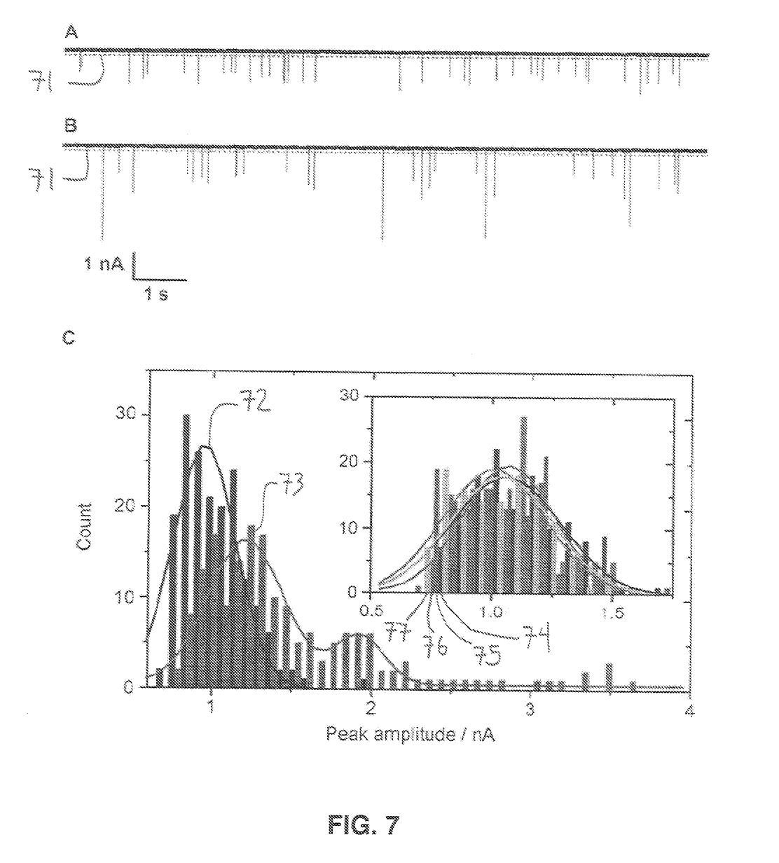

At the beginning of each experiment, the response of the submicrometer pore-based Coulter counter to single virions was characterized. See Example 10 for a detailed analysis of the bandwidth of the measurement, the bandwidth and sampling frequency required to resolve an event due to a virus completely, and the effects of digital filtering and decimation of data on the peak amplitudes and half-widths of the events. Even in the absence of antibodies, PBCV-1 virions passing through the conical pore created resistive pulses with peak amplitudes significantly above the baseline noise (FIGS. 6 and 7). These pulses were analyzed with a computer algorithm by using a threshold value for the peak amplitude to identify individual “virus events” (the dotted red line in FIGS. 7A and B indicates the threshold value); peaks that had at least 10 times the amplitude of the standard deviation of the current noise from its mean baseline value (root mean square current noise, here called RMS noise) were counted as viruses (most events generated from a solution containing only virus had peak amplitudes of at least 700 pA, or ≈40 times the RMS current noise). The analysis of resistive pulses showed that the frequency of events was proportional to the concentration of the virus in a concentration range from 4.4×10⁷ to 2.5×10⁹ particles/mL; The following relationship was found: frequency of events [Hz] 4.0×10⁻⁹ [HzmL particles⁻¹]× concentration of virus particles [particles/mL]; N=6; R²=0.95. See also the disclosure in Example 11.

FIG. 7 shows the detection of an antibody-virus interaction using a submicrometer pore. The panels show: A) Current versus time trace before addition of antiserum: The transient increases in resistance (events) that occurred when viruses passed through the pore led to transient reductions in current. The dotted line 71 represents the threshold used to distinguish events caused by the passage of viruses from current noise. B) Current versus time trace approximately 8 min after addition of antiserum: The mean peak amplitude was approximately 22% larger than the mean peak amplitude before addition of antiserum, whereas the four largest peaks were presumably due to aggregates of virus particles. C) Histograms of the peak amplitudes of 175 events that occurred before antibody binding (shown in black at 72) and 6-8 min (shown in grey at 73) after addition of antiserum (final virus concentration 4.4×10⁸ particles/mL, final dilution of the antiserum: 0.001× the original antiserum). The Gaussian mean of the first (bigger) peak in the grey histogram 73 shifted compared to the histogram before antibody binding (shown in black at 72). The second peak in the grey histogram 73 occurred presumably due to the formation of dimers. The inset represents data from control experiments; the histograms show events that occurred before (74) and 2.5-3.5 min (75), 7.5-8.5 min (76), and 13-15 min (77) after addition of serum from a rabbit that was not immunized (final virus concentration 4.4×10⁸ particles/mL, final dilution of this control serum: 0.0013× the original control serum). The inset histogram includes repeating data sets of reference numerals 74, 75, 76, and 77, in blocks of four, respectively. The change in the mean peak amplitude of the control experiments was <6.5%.

In order to estimate the size of individual virus particles without any bound antibody, approximately 1400 virus events were analyzed. This analysis was based on fitting a Gaussian distribution to a histogram of the peak amplitudes as shown in FIG. 7C. Applying the linear relationship between peak amplitude and particle volume to the mean peak amplitude from the Gaussian distribution then made it possible to calculate the mean volume of the virus particles. Using equations that relate the volume of an icosahedron to its diameter, a diameter of 203±14 nm was obtained along the five-fold axes for PBCV-1 virions. This result compares well with measurements of the size of PBCV-1 by cryoelectron microscopy, which revealed an average diameter of 190 nm along the fivefold axes (depending on the microscope technique, diameters of 140-190 nm have been reported; however, cryo-electron microscopy is known to preserve the native state of the virus and may therefore reflect the size of the virus particles in their hydrated state more accurately than EM techniques that require drying of the samples). Coulter counting with a submicrometer pore is thus a rapid, simple, and effective technique to determine the size of virus particles in their native state.

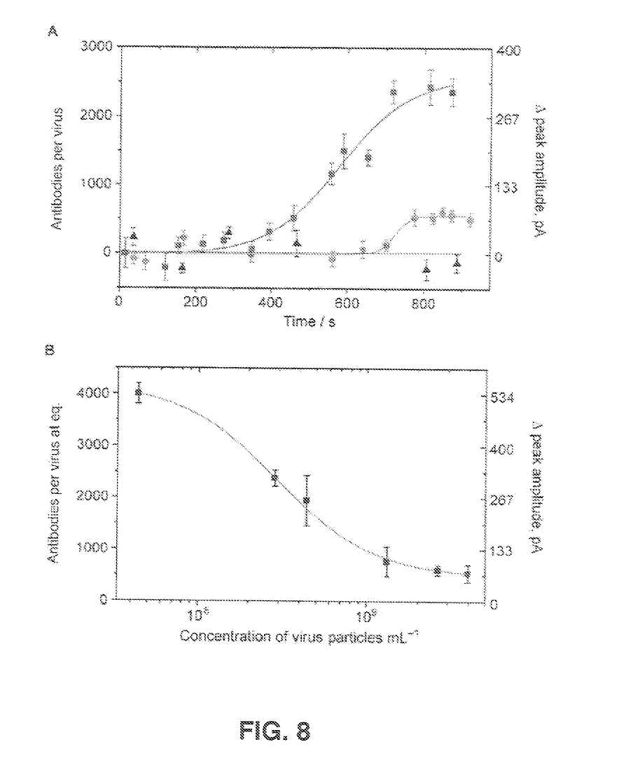

To examine the binding of antibodies to PBCV-1, the peak amplitude of the events after adding a polyclonal antiserum against PBCV-1 was monitored; the dilution of the antiserum and therefore the concentration of antibodies in the mixture was kept constant in all experiments while the concentration of virus particles was varied (the concentration of the specific antibody in the antiserum was unknown; however, the methods disclosed in Example 12 calculated a lower boundary of 0.55 mg/mL for the concentration of the specific antibody based on the collected data). Upon addition of antiserum to solutions with various virus concentrations, the peak amplitudes of the virus events increased. A Gaussian fit of the resulting histograms showed a shift of the mean peak amplitude that indicated particles of increased volume (FIG. 7C). The final increase in amplitude upon antibody binding onto individual virus particles ranged from +7 to +60% (FIG. 8), depending on the ratio of antibody concentration to virus concentration in the solution. By calculating the difference between the mean current peak amplitudes from the Gaussian fits before and after addition of antiserum (FIG. 7C), the increase in volume due to antibody binding was determined. The maximum increase in volume occurred at the highest antibody to virus ratio and was +1.4×10⁶ nm³, corresponding to +60% (FIG. 6).

FIG. 8 depicts the kinetics of antibody binding at different ratios of antibody to virus concentration and estimation of the maximum number of antibodies that can bind to the virus. In these experiments, the final dilution of the antiserum or control serum was held constant at 0.001× the original serum. The panels show: A)

Plot of the number of antibodies bound to virus particles versus time. The final concentration of the virus was either 2.8×10⁸ (squares) or 4.0×10⁹ particles/mL (circles). The triangles represent a control experiment with nonspecific rabbit serum and a virus concentration of 3.4×10⁸ particles/mL. The error bars reflect the error of the mean value from a Gaussian fit to a histogram of the peak amplitudes of at least 50 events. B) Plot of the number of antibodies bound to PBCV-1 viruses at equilibrium versus the concentration of the virus (increasing virus concentration corresponds to decreasing antibody-to-virus ratio). The data were fitted with a sigmoidal function of the form y=A2+(A1×A2)/p(1+x/xₒ); N=6, R²=0.99. The error bars were calculated by summing the standard deviation of the mean values of the Gaussian fits to histograms of the peak amplitudes.

FIG. 7C also shows a second peak in the histogram of the peak amplitudes upon addition of antibody to the virus particles. The mean value of the Gaussian distribution fitted to the second peak was approximately twice that of the first peak. Since the antiserum that was used can cause aggregation of viruses (see also Example 13), the second peak may be caused by dimers of viruses that were linked by the divalent polyclonal IgG antibodies in the antiserum. Control experiments with serum from a rabbit that was not immunized caused only a small (<6.5%) change of the mean of a Gaussian fit to the peak amplitudes of the virus (FIG. 7C, inset), indicating that binding of nonspecific antibodies (or other proteins) to the viruses was minimal.

Using the aforementioned approach to calculate the increase in volume of virus particles upon binding of antibodies, estimate the number of antibodies attached to individual virus particles was estimated by assuming that each antibody contributed a volume of 347 nm³ (this molecular volume of IgG antibodies was measured by atomic force microscopy). Since the assay presented here provided the ability to record virus events continuously, it was possible to follow the number of antibodies bound to virus particles over time. It was thus possible to extract the kinetics of the antibody-virus interaction at different ratios of antibody concentration to virus concentration as shown in FIG. 8A. The equilibrium stage of antibody binding was typically reached after 6-13 min (depending on the ratio of antibody to virus). The equilibrium occupancy was found to decrease with decreasing antibody-to-virus ratio and that it ranged from 500 to 4,000 antibodies per virus particle (FIG. 8B).

Based on the sigmoidal fit of the data shown in FIG. 8B, the maximum number of antibodies that could bind to the virus particles was estimated at 4,200±450. PBCV-1 is known to contain a major capsid protein which carries the primary epitope to which the polyclonal antiserum binds. PBCV-1 is enclosed in 5040 copies of this major capsid protein. Since the observed maximum number is close to the protein copy number, namely 4,200±450 antibodies bound to each virus particle, most of these primary epitopes were accessible for antibody binding. The close agreement of these numbers also suggests that the majority of the antibodies in the antiserum were bound to an individual virus particle via one of their two binding sites (i.e., monovalent binding; purely divalent binding would result in a maximum possible antibody load of ≈2520 per virus assuming that the major surface antigen is responsible for most antibody-binding interactions). The observation that this antiserum aggregates the virus also supports the hypothesis of significant monovalent binding.

Thus, the present technology may determine the number of antibodies bound to viruses in their native conformation. The present method is label-free, nondestructive, requires no immobilization or modification of the virus or antibody, and can establish if the antibody is suitable for immunoprecipitation. Decreasing the diameter of the pore may allow the detection of virus-antibody interactions for viruses that have diameters less than 190 nm. Due to the specificity of most antibody-virus interactions, this method may be used to detect the presence of an antibody directed against a particular virus in complex media such as serum (here the anti-PBCV-1 antibody); it may therefore be useful for immunoassays and vaccine development.

For example, the ability to determine the number of antibodies bound to a virus enables at least three important applications. First, it makes it possible to predict the efficacy of antibody-mediated neutralization of viruses. Second, the number of antibodies that are bound to a virus can be used for determining the antibody's affinity and the valency of binding. And third, antibodies binding to a virus particle represent an accessible example of a well-defined self-assembly; monitoring this assembly process may thus be useful as a model system for studying templated self-assembly. Such a system may promote other attempts of controlled nanoassemblies (e.g., fabrication of hierarchical nanostructures through the binding of nanoparticles to engineered templates).

Aspects of present technology also include using resistive-pulse sensing to estimate i), the affinity constant between one object and another object, and ii) the solid phase affinity constant between one object and another object. For example, a method described herein illustrates how the solid phase affinity constant of an antibody for its antigen immobilized on a particle may be determined using a micropore and the resistive-pulse technique. This method can be directly applied to resistive-pulse sensors that use submicrometer pores or nanopores. Submicrometer pores and nanopores, which may be used to detect individual proteins, also enable this method to be readily adapted to measuring the affinity constant of monovalent interactions including, but not limited to: Fab fragments binding to a virus particle, a monovalent ligand binding to a monovalent or polyvalent protein, a monoclonal antibody binding to a monovalent antigen, and nanoparticies (unmodified particles and particles modified with functional groups) binding to a synthetic or biological object (template). These pores also allow this method to be readily adapted to measuring the avidity constant of polyvalent interactions including but not limited to: polyclonal antibodies binding to antigens in solution, monoclonal antibodies binding to proteins in solution that have than one copy of the epitope, polyclonal or monoclonal antibodies binding to viruses, and nanoparticles binding to a synthetic or biological object (unmodified particles and particles modified with functional groups).

An immunoassay using a micropore and the resistive-pulse method may be used to detect the interaction of a monoclonal anti-streptavidin antibody with streptavidin-functionalized nanoparticles. Resistive-pulses can be recorded during the passage of these spherical colloids with a diameter of 510 nm through the pore. A constant concentration of colloids (1.2×10⁹ particles mL⁻¹) can be incubated with an increasing concentration of the antibody, and the resistive-pulses from the colloids with bound antibody can be recorded and compared to the pulses before antibody binding. The diameter of the colloids at different concentrations of antibody can be estimated using the following equation since the pore had cylindrical geometry:

$$\vert \frac{δ I}{I} \vert = \frac{D}{L} [ \frac{arc sin ( d / D )}{\sqrt{1 - {( d / D )}^{2}}} - \frac{d}{D} ] \qquad ( 1 )$$

where δI is the change in current from baseline, I is the baseline current flowing through the pore, D is the diameter of the pore, L is the length of the pore, and d is the diameter of the colloid. Since the change in current from baseline increased with increasing concentration of antibody, the diameters of the nanoparticles appear to increase due to antibody binding. Assuming that the antibody-antigen interaction reached equilibrium (binding of polyclonal antibodies to virus particles typically reaches equilibrium within 13 min), it is possible to use resistive-pulse sensing to estimate here the solid phase affinity constants of e.g. antibody-antigen interactions.

Eq. (1) is based on spherical particles by proposing that the transient increase in the resistance of the pore is due to the displacement of a volume of conducting electrolyte by the spherical particle. By using Eq. (1), it may be assumed that binding of antibodies to the colloids created a dielectric layer of antibodies which could be treated as an increase in the diameter of the colloids. The thickness of this hypothetical confluent film (and therefore the mean increase in particle diameter) may depend on the extent of coverage of the colloids with antibodies.

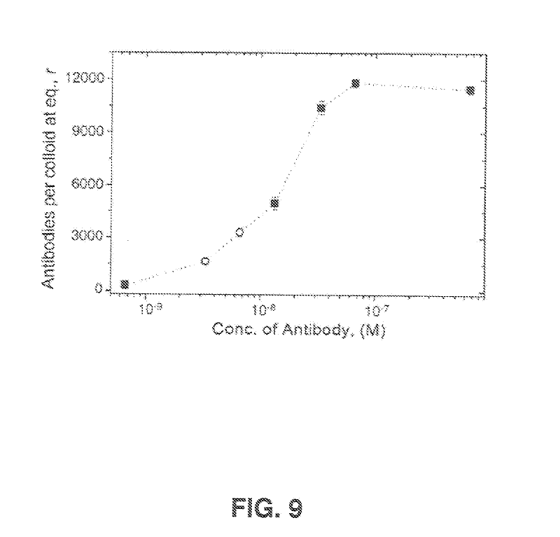

The present technology can incorporate the colloid diameters from Eq. (1) and can yield additional information if they are used to calculate the volume of these particles. Basing the analysis on particle volumes has the additional benefit of extending it to particles that may not be perfect spheres; irregularly (spheroidal) shaped particles of the same volume appear to produce peak amplitudes of identical magnitude. Based on the diameters of the colloids data, the corresponding volumes of spheres with these diameters (using V=⅙πd³) may be calculated in order to obtain the volume of the colloids with and without antibodies bound. By assuming a volume of 347 nm³ for an antibody, the number of antibodies bound to each colloid at equilibrium may be calculated; r as shown in FIG. 9. This additional information enabled the estimation of the solid phase affinity constant of the antibody as demonstrated below.

FIG. 9 shows the number of monoclonal anti-streptavidin antibodies from mouse bound per streptavidin-functionalized colloid, r, vs. the initial concentration of antibody in solution. The concentration of the colloids was held constant at 1.2×10⁹ particles mL⁻¹. This plot is based in part on analyzing and converting the data presented by Saleh, O. A., Sohn, L. L., 2003. Proc. Natl. Acad. Sci. U.S.A. 100, 820-824, which is incorporated herein by reference.

The derivation of Eq. (1) did not take into account possible effects from the surface charge of the particle. Recent Coulter counting experiments with double stranded DNA (dsDNA) demonstrated that the highly charged properties of dsDNA can significantly alter the peak amplitude of the resistive-pulse. Depending on the ionic strength of the buffer, dsDNA even caused transient increases in current (conductive-pulses) when it passed through a nanopore. It has been proposed that the amplitude and sign of the pulse is determined by two competing effects. One, the dsDNA displaced a volume of conducting electrolyte thereby removing mobile charge carriers from the electrolyte solution in the pore which caused a transient increase in resistance. Two, the dsDNA delivered a cloud of mobile counter ions into the pore due to its highly charged nature (two negative charges per base pair) which caused a transient decrease in resistance. Under certain conditions, these two effects are able to cancel each other out causing the passage of dsDNA through the pore to create no signal.

The effect due to the surface charge of the dsDNA depends on the length of the dsDNA and the length of the pore; if the length of the dsDNA is significantly shorter than the length of the pore, the effect of the surface charge on the amplitude of the resistive pulses is assumed to be negligible. It has also been demonstrated that particles with nearly identical diameters of ˜60 nm and different surface charge (particles with 120 carboxylic acid groups and particles with 24,200 carboxylic acid groups in an aqueous electrolyte with pH 7.3, where most of these carboxylic acid groups were deprotonated and thus charged) produced resistive-pulses with nearly identical peak amplitudes in pores with lengths ≧0.83 μm (ratio of pore length to particle diameter of 830 nm/60 nm=13.8). In other reports, the length of the pore was 7-9 μm and the diameter of the colloids was 510 nm. These values constituted experimental conditions that were nearly identical to other reports using a ratio of pore length to particle diameter of 7000 nm/520 nm=13.5. Based on the data, the surface charge of the antibody-colloid complex does not appear to significantly affect the peak amplitude of the resistive pulse.

Before estimating the solid phase affinity constant, the valency of the antibody-antigen interaction must be considered. Given that the antigen was immobilized on the colloid, the antibody could bind in a monovalent or divalent fashion (i.e. one or two arms of the antibody could bind to streptavidin molecules). Both possibilities may be investigated as follows.

In order to estimate the solid phase affinity constant, Kₐ, of the antibody in the case of monovalent binding between the antibodies and the antigen at all concentrations of antibody, the binding equilibria of the antibody-antigen interaction studied by Saleh and Sohn was analyzed. Under these conditions, the colloids (with many antigen molecules covalently attached to their surface) may be considered analogous to macromolecules that possess many identical binding sites for a single ligand. The thermodynamics of such as system are straightforward. The derivation begins with the simplest case, the one in which the entire macromolecule possesses only a single binding site for the ligand. This situation is equivalent to the interaction of an antibody, Ab, with a colloid, C_{Ag} that would carry a single antigen (here streptavidin) molecule. This scenario can be described using the following equation:
Ab+C_{Ag}=C_{Ag}Ab. (2)
The equilibrium for this reaction is characterized by Kₐ, the equilibrium constant, which in this example represents the solid phase affinity constant of the antibody (in general, this equation represents the affinity of one object for another):

$${K}_{a} = \frac{[ {C}_{Ag} Ab ]}{[ {C}_{Ag} ] [ Ab ]} , \qquad ( 3 )$$

where [C_{Ag}Ab] represents the concentration of the complex between the antigen-functionalized colloid and the antibody at equilibrium, [C_{Ag}] represents the concentration of free colloids at equilibrium, and [Ab] represents the concentration of free antibody at equilibrium. The binding equilibria governed by Eq. (3) can be characterized by a binding isotherm of the for:

$$r = \frac{[ {C}_{Ag} Ab ]}{[ {C}_{Ag} ] + [ {C}_{Ag} Ab ]} = \frac{{K}_{a} [ Ab ]}{1 + {K}_{a} [ Ab ]} , \qquad ( 4 )$$

where r represents the moles of antibody bound per mole of colloid at equilibrium (or the number of antibodies bound per colloid at equilibrium). Eq. (4) describes a single interaction and can be extended to colloids with multiple antigens (in analogy to macromolecules with multiple binding sites) by adding the isotherms for all interactions:

$$r = \frac{{nK}_{a} [ Ab ]}{1 + {K}_{a} [ Ab ]} , \qquad ( 5 )$$

where n represents the number of antigens immobilized on a colloid. Provided that r and [Ab] are known or can be determined experimentally, Eq. (5) can be used to determine Kₐ, and n.

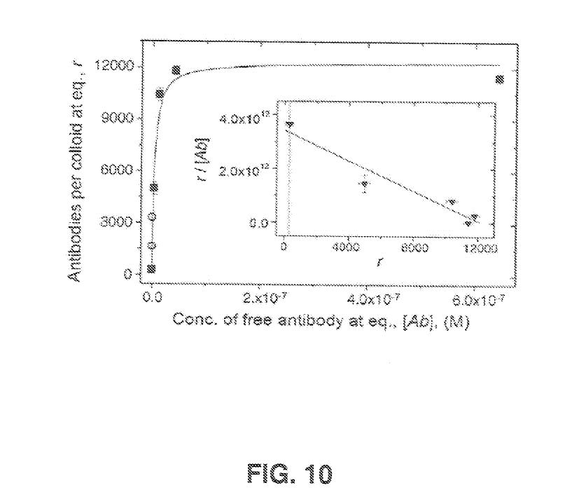

The concentration of the colloids is typically known and was held constant at 1.2×10⁹ particles mL⁻¹ by Saleh and Sohn in all experiments (the concentration of the colloids may also be determined by the frequency of the resistive pulses). The volume-based analysis of the Coulter counting data introduced in the present disclosure made it possible to calculate the number of antibodies bound per colloid at equilibrium, r, as shown in FIG. 9. Multiplying r by the concentration of colloids revealed the concentration of bound antibodies at equilibrium. The concentration of free antibody at equilibrium, [Ab], was then obtained by subtracting the equilibrium concentration of bound antibodies from the initial antibody concentration. During this analysis, it was observed that two of the colloid diameters reported by Saleh and Sohn corresponded to bound antibody concentrations that exceeded slightly (≦56%) the initial antibody concentration. Since it is not possible that more antibodies bound to the colloids than were present in solution, the lower limit of the mean diameter of the antibody-decorated colloids, which was provided by the error bars in Saleh and Sohn's paper is used to calculate the number of antibodies bound and hence to obtain a plausible concentration of bound antibody. These two data points are denoted in FIGS. 9 and 10 with open circles as opposed to filled squares, and were excluded from the generation of the best fits in FIG. 10.

FIG. 10 shows a graphical plot of the number of antibodies bound per colloid at equilibrium, r, as a function of the free antibody concentration at equilibrium, [Ab]. Eq. (5) was fitted to the data using nonlinear regression (R²=0.98, N=5). The two data points marked by open circles were not included in the Scatchard plot and they were not included in the best fit analysis (see main text). The inset represents a Scatchard plot of r [Ab]⁻¹ vs. r (Eq. (6)). Linear regression was used to fit the data (R²=0.93, N=5).

Based on the analysis derived above, it is possible to plot the number of antibodies bound per colloid at equilibrium, r, as a function of the free antibody concentration, [Ab] (FIG. 10). By fitting the data with Eq. (5) (R²=0.98, N=5), the maximum number of antibodies which could bind to the colloids at saturation, n, can be obtained and the solid phase affinity constant, Kₐ, for the interaction. As expected, the value of n=12,300±730 obtained from the fit was in good agreement with the n (11,800) calculated above (FIG. 1) from the maximum volume increase as determined from resistive-pulses, and this value also matches well with the number of 9800 streptavidin molecules immobilized on the colloid (Saleh and Sohn, 2003a). Therefore, at saturation, approximately one antibody was bound per streptavidin molecule (monovalent binding). A value of 2.6×10⁸±0.8×10⁸ M⁻¹ was obtained for Kₐ from this analysis, which is in agreement with the manufacturer's specifications that this monoclonal antibody has an affinity greater than 1×10⁸ M⁻¹. Taking possible ligand depletion effects into account resulted in a solid phase affinity constant of Kₐ=3.7×10⁸±1.9×10⁸ M⁻¹.

In order to examine the case of bivalent binding, the data can be plotted using the following linearized form of Eq. (5):

$$\frac{r}{[ Ab ]} = {nK}_{a} - {rK}_{a} . \qquad ( 6 )$$

Eq. (6) is known as the Scatchard equation and the inset of FIG. 10 shows a plot of the data in this format (this plot makes it possible to determine Kₐ by linear regression; and so obtained Kₐ=2.8×10⁸±0.4×10⁸ M⁻¹). Scatchard plots, as shown in FIG. 10, are commonly used to assess the presence of divalent binding. If there was significant divalent binding, the Scatchard plot would be non-linear since at least two apparent solid phase affinity constants would determine the binding interaction. The data in FIG. 10, inset, follow a linear trend which implies predominantly monovalent binding across all antibody concentrations; however, the error bars in the horizontal and vertical direction combined with the debate on the validity of using Scatchard plots for determining the valency of binding makes a conclusive determination of the valency of binding impossible. It is therefore conceivable that a fraction of antibodies bound divalently under conditions of low antibody concentration (i.e. at low occupancy of antigens by antibodies). As a consequence, the solid phase affinity constant of 2.6×10⁸ to 3.7×10⁸ M⁻¹ obtained here is most accurate for the condition of significant occupancy of antigens by antibodies.

It is also possible that a fraction of the antistreptavidin antibody bound to the colloids in a non-specific fashion. Other calculations have shown that the maximum number of antibodies bound to a colloid was 11,800, while the manufacturer specifies ˜9,800 streptavidin molecules on the surface of the colloids. This difference suggests that as many as 17% of the antibodies may have been bound non-specifically. Assuming that the fraction of antibodies that bound non-specifically remained at a constant 17% over the range of antibody concentrations used, re-calculating the solid phase affinity constant for the anti-streptavidin antibody using Eq. (5); gives the resulting solid phase affinity constant 1.8×10⁸±0.7×10⁸ M⁻¹.

The present technology, therefore, can use resistive-pulse sensing to estimate the solid phase affinity constant for the binding of an antibody to a specific antigen or more generally the affinity constant for receptor-ligand interactions when the binding interaction is predominately monovalent (e.g. a monovalent ligand and a monovalent or polyvalent protein or receptor). The system analyzed here is analogous to antibodies binding to intact virus particles or to the attachment of nanoparticles to templates (or other objects such as viruses). The quantitative approach of the present disclosure makes it possible to estimate the solid phase affinity or the avidity constant of monoclonal antibodies or the affinity of Fab fragments for their binding to antigens on viruses (or other antigens that are intrinsically immobilized on nanoparticles or even floating in solution) in physiological conformation. In addition, this method may be used for determining the number of nanoparticles attached to another object (e.g a biological or synthetic template) and thus for extracting the average “association constant” of nanoparticle-template interactions. Obtaining these values for synthetic systems is difficult by established methods such as electron microscopy or atomic force microscopy. The present technology can therefore find use in characterization and fabrication of the next generation of functionally assembled nanodevices.

In yet another aspect of the present technology, a submicrometer pore or nanopore-based assay may be used for detecting the formation of protein aggregates and crystals in solution. Application of the present methods may monitor in realtime the formation of assemblies of protein and simple analysis of the data may determine whether or not protein crystals formed. Currently, light scattering techniques are commonly used for studying various aspects of protein aggregation and crystal growth; however, due to the nature of this bulk measurement, it is not possible to determine a true distribution of protein assemblies from light scattering data. In contrast, resistive-pulse sensors detect individual particles and can therefore provide the true distribution of the protein assembles. This true distribution may be used for rapid determination of whether or not protein crystals formed under the given conditions. Due to the small footprint of nanopores and the low power, cost, and reagent requirements, the present methods and assays could be used in high-throughput or laboratory applications for drug discovery and protein research.

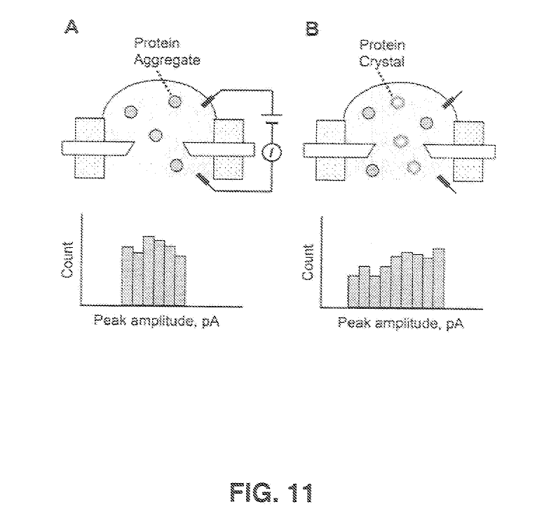

Methods include the following features. The candidate solution for generating protein crystals is prepared and placed in a microliter-volume fluidic well (affording short mixing time and minimal reagent costs) that contains the nanopore. From this starting point, there are three possible outcomes: (i) the solution does not generate any protein aggregates or crystals; (ii) the solution generates protein aggregates which are detected by the nanopore and possess a specific distribution as shown in FIG. 11A; and (iii) the solution generates protein aggregates and crystals which are detected by the nanopore; the resulting distribution is expected to be different from protein aggregates alone as shown in FIG. 11B.

The assay proposed here allows integrating structure-based drug design based on protein crystallization with high-throughput screening techniques. The combination of these two techniques provides a powerful drug search paradigm for pharmaceutical companies and increases their probability of finding leads for a target protein. In addition, the present technology will yield the solubility of proteins. Solubility is an important parameter for the pharmaceutical industry and the exact same nanopore-based solubility assay can be applied to small molecule therapeutics.

### EXAMPLE 1

Nanomachining using a femto-second pulsed laser. A cover glass (Corning 0211 borosilicate, Fisher Scientific, Pittsburgh, Pa.) was fixed to a 3-axes microscope nanomanipulation stage (Mad City Labs, Inc., Madison, Wis.). A few drops of water were placed on the upper side of the cover glass at the area that was to be machined (if the machining time was greater than 30 minutes, an aluminum compartment sealed with tape was used to minimize evaporation of water). The laser, a directly diode-pumped Nd:glass CPA laser system (Intralase Corp., Irvine, Calif.), was focused through the a 100× oil immersion microscope objective (N.A.=1.3, Zeiss, Thornwood, N.Y.) and the cover glass to the machining site (FIG. 1a). Pulses were used with a duration of 600-800 fs (femtoseconds) that were frequency doubled from 1053 nm to 527 nm. The glass was machined by scanning the laser in circular patterns which removed material layer by layer. Since the subsequent layer was formed under water, machining always proceeded at the glasswater interface. The submicron pores were machined in a three stage process. The following parameters were used for the pores: 35 μm cylinder machined with 60-80 nJ per pulse at a frequency of 1.5 kHz; wide part of the cone machined with 12-15 nJ per pulse at 1.5 kHz; tip of the cone machined with 8-13 nJ per pulse at 10 Hz. After machining, the glass coverslides were left in water for 12 hours with the 35 μm cylinder facing down; this configuration facilitated settling of debris out of the pore. The glass coverslides were cleaned in a fresh mixture of 3:1 concentrated sulfuric acid to 30% hydrogen peroxide for at least 15 minutes, prior to use.

### EXAMPLE 2

Peak amplitude is proportional to the volume of spherical particles in submicron pores with conical geometry.

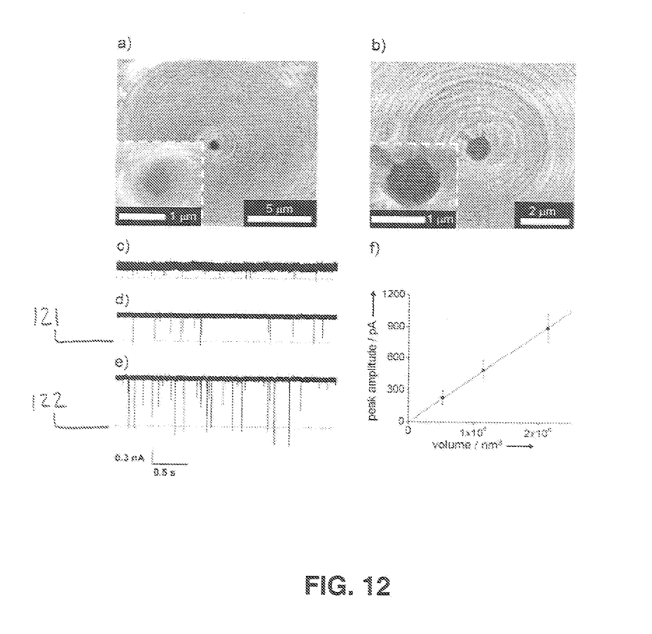

FIG. 12 shows SEM images of conical pores with diameters of 575 and 900 nm and determination of the relationship between peak amplitude and particle volume in conical pores with submicron diameter. The panels show: a) SEM image looking into the 35 μm cylinder of the pore with a diameter of 575 nm. The inset shows a close-up of the narrowest part of the pore. b) SEM image looking into the 35 μm cylinder of the pore with a diameter of 900 nm. The inset shows a close-up of the narrowest part of the pore. c) Current versus time trace of particles with a diameter of 100 nm passing through the pore shown in a). The doffed line represents the mean current amplitude of the 9 peaks d) Current versus time trace of particles with a diameter of 100 and 130 nm (mean current amplitude from the 130 nm particles is shown at 121) passing through the pore shown in a). e) Current versus time trace of particles with a diameter of 100, 130, and 160 nm (mean current amplitude from the 160 nm particles is shown at 122) passing through the pore shown in a). f) Plot of the average peak amplitude of the resistive-pulses caused by particles with a diameter of 100, 130, and 160 nm passing through the pore shown in a) versus particle volume. The data were fitted using a linear regression algorithm that required the line to pass through the origin; the slope of the line was 4.2×10⁻⁴ pA nm⁻³. The slope was 3.9×10⁻⁴ pA nm⁻³ for the pore with a diameter of 650 nm (FIG. 1b, c).

SEM sample preparation. The glass coverslides were coated in gold (thickness ˜10 nm) using a sputter coater (Structure Probe Incorporated, West Chester, Pa.) and imaged with a high resolution scanning electron microscope (FEI Company NOVA 200 Nanolab, Hillsboro, Oreg.). See FIGS. 12a and b. After imaging, the gold layer was removed using a 3:1 mixture of fuming nitric acid and concentrated hydrochloric acid.

### EXAMPLE 3

Experimental Section. Solutions: All solutions were prepared with deionized water (resistivity of 18.2 MΩcm, Aqua Solutions, Jasper, Ga.) and potassium chloride, sulfuric acid (both from EMD Biosciences, La Jolla, Calif.), TRIS (Shelton Scientific, Shelton, Conn.), bovine serum albumin (Sigma, St. Louis, Mo.), Tween 20 (Mallinckrodt Chemicals, Phillipsburg, N.J.), hydrochloric acid (VWR International, West Chester, Pa.), nitric acid (Fluka Chemie, Buchs, Switzerland), and hydrogen peroxide (EMD Chemicals, Gibbstown, N.J.) were used without further purification. Recording buffer was filtered through sterile 0.1 or 0.2 μm, low protein absorption polyethersulfone membrane filters (both from Pall, East Hills, N.Y.). Affinity-purified monoclonal antibody from mouse against baculovirus envelope gp64 protein (eBioscience, San Diego, Calif.) was used without further modification. Affinity-purified goat anti-mouse antibody (H+L) conjugate labeled with tetramethylrhodamine isothiocyanate (TRITC) and affinity-purified goat anti-rabbit antibody (H+L) conjugate labeled with TRITC (Zymed, San Francisco, Calif.) were diluted in recording buffer and filtered through either a 0.1 μm or 0.2 μm membrane filter. The TRITC labels on these antibodies were not used for the submicron pore assays but were useful to perform control experiments of immunoprecipitation with fluorescence microscopy (see Figure S4). The sheep anti-SEB serum and purified SEB (both from Toxin Technology, Sarasota, Fla.) were filtered through a 0.1 μm membrane filter. The 100, 130, and 160 nm particles (polystyrene microspheres functionalized with carboxyl groups, Bangs Laboratories, Fishers, Ind.) were used at a concentration of ˜1×10¹⁰ particles mL⁻¹ in recording buffer.

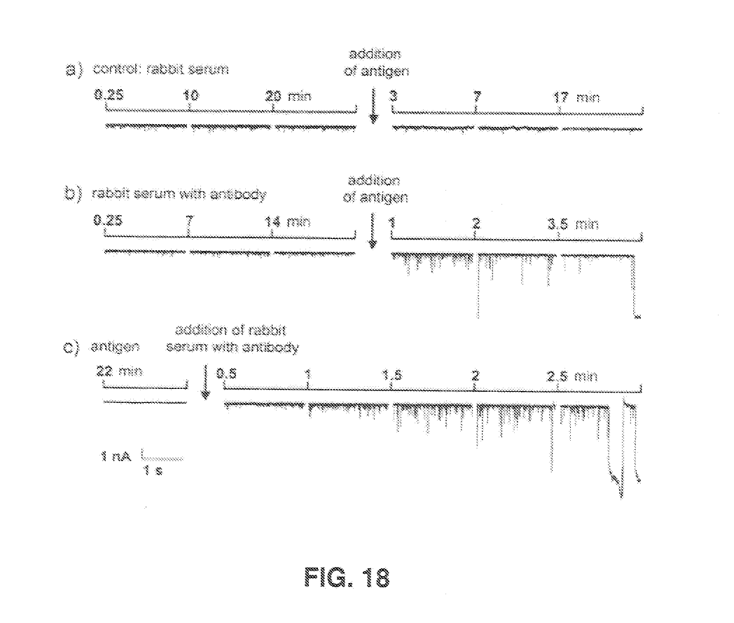

Data acquisition. The glass coverslide was placed (narrowest part of the submicron pore facing the top liquid compartment) on a fluidic channel in poly(dimethylsiloxane) (PDMS, Sylgard 184 Silicone, Dow Corning, Midland, Mich.). A fresh film of PDMS with a hole in the center was placed on the top of the glass coverslide (FIG. 2) to confine the electrolyte (recording buffer) to the top side of the cover glass. In order to guarantee reliable recording conditions while measuring relatively large currents (100-180 nA), Ag/AgCl pellet electrodes were used (Eastern Scientific, Rockville, Md.). A patch clamp amplifier was used (Axopatch 200B, voltage clamp mode, applied potential of either 0.2 V (FIG. 3, FIG. 18) or 0.15 V (FIG. 4), analog low-pass filter set to a 100 kHz cutoff frequency), a low noise digitizer (Digidata 1322, sampling frequency set to 500 kHz), and a computer with recording software (Clampex 9.2, all from Axon Instruments, Union City, Calif.) for data acquisition.

Since the current was recorded at high bandwidth (˜50 kHz), care was taken in the analysis of the data to avoid two possible problems: amplifier saturation, and recording digitized data with low signal-to-noise ratios (SNR<1). Amplifier saturation was avoided by ensuring that the currents including their high-bandwidth noise were at all times within the dynamic range of the amplifier and of the digitizer used. The maximum recorded current with its RMS noise of ±0.06 nA was at all times below 180 nA; the dynamic range of the recording setup was ±200 nA. The second problem, recording digitized data with low SNRs, occurs if the amplitude of the signal of interest is considerably lower than the noise levels. Such low amplitude resolution can lead to inaccuracies during off-line analysis (e.g., event detection after filtering). This condition was, however, avoided since the lowest signal-to-noise ratio of the high bandwidth (˜50 kHz) data recorded was 2:1 (peak amplitude:RMS noise). In addition, after filtering with a cutoff frequency of 10 kHz, only peak amplitudes of at least 5 times the RMS noise were included in the quantitative analysis of immune complexes (see below).

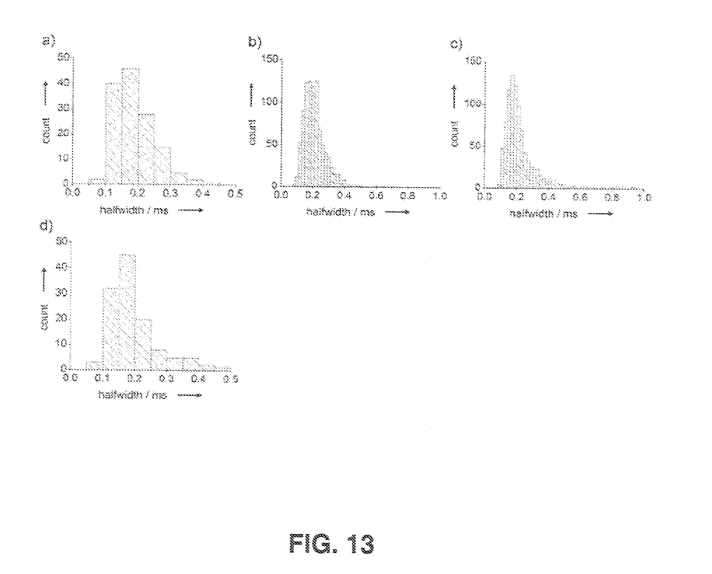

Data processing and event detection. For all data processing and event detection, Clampfit 9.2 (Axon Instruments, Union City, Calif.) was used. The recorded data was filtered with a digital Gaussian low-pass filter with a cutoff frequency of 10 kHz and then decimated it to a sampling frequency of 50 kHz. A threshold search was used with two criteria to identify events (event is defined here as an object, such as an immune complex or nanoparticle, passing through the pore). Transient reductions in current were only counted as an event if these reductions had an amplitude of at least 5 times the RMS noise of the current trace for a duration of at least 25 μs. These criteria established the lower limits for the algorithm that we used for analysis to distinguish events from noise; these criteria do not imply that most events lasted only for 25 μs. In fact, the vast majority of events lasted at least 100 μs. For instance, the histograms in FIG. 13 show that the mean halfwidth of the smallest immune complexes was 190±60 μs and the mean halfwidth of the smallest nanoparticles was 210±110 μs. Thus, the sampling intervals of 20 μs used were sufficiently short to resolve the events.

Shown in FIG. 13 are histograms of the halfwidths of events caused by immune complexes and nanoparticles passing through submicron pores with conical geometry. The panels show: a) Histogram of the halfwidths of some of the smallest events (mean peak amplitude=107±16 pA) that are shown in FIG. 5d (recorded during the interval 4.8-6.9 min of the experiment). b) Histogram of halfwidths of medium-sized events (mean peak amplitude=122±40 pA) that were recorded during the interval 2.5-4.0 min of the experiment shown in FIG. 5a. c) Histogram of the halfwidths of large events (mean peak amplitude=380±430 pA) that were recorded during the interval 8.3-8.6 min of the experiment shown in FIG. 5a. d) Histogram of the halfwidths of events caused by the smallest nanoparticles (diameter 100 nm) moving through the pore with a diameter of 575 nm (FIG. 12).

The collected data of events were analyzed using Origin 7.5 software (OriginLab, Northampton, Mass.). The smallest peaks that were collected by the threshold search had a dl/l value of 0.1%. A stringent requirement of a threshold of 5 times RMS was applied for detection of events to ensure accurate assignment. dl/l values of 0.1% have also been reported for reliable detection of DNA and nanoparticles.

Electrical current noise of submicron pores with conical geometry. The noise values reported in Table 1 are considerably higher than reported values for planar lipid bilayer (PLB) experiments (e.g. PLB experiments report a current noise of <1 pA RMS at a bandwidth of 3 kHz). This discrepancy is due to the large difference in resistance between the submicron pores used (1-2 MΩ) and PLBs (>10 GΩ). The large resistance of PLBs leads to currents in the pA range, and under these low-current conditions the noise of the recordings is dominated by two sources: 1) the thermal voltage noise of the access resistance i_{rc} and 2) the noise resulting from the interaction of the headstage noise with the input capacitance i_{vc}. In the present disclosure, a planar lipid bilayer was not formed over the submicrometer pores and consequently the resistance values were significantly lower than pores that have a bilayer on them. Under these conditions, the thermal shot noise, iₜₕ, dominates. Thermal noise is defined as:

$${i}_{th} = \sqrt{\frac{4 kTB}{R}} \qquad ( 7 )$$

where k is the Boltzmann's constant, 1.38·10⁻²³ m² kg s⁻² K⁻¹, T is the temperature in kelvin, R is the resistance of the pore, and B is the bandwidth. With the experimental values of R=1.4·10⁶Ω, T=294 kelvin, and B≈10,650 Hz, the theoretically expected RMS noise was ˜11 pA, which is somewhat lower than the value that is reported in Table 1. The noise recorded was most likely somewhat higher than the theoretical expectation due to other sources of noise such as amplifier noise and dielectric noise. In any event, the experimentally recorded RMS noise value of 16 pA was low when considering the “large” current of 140 nA during the recordings in the present work. This low noise value confirms that the design of the pores and the material properties of the glass substrate are well-suited for Coulter counting of nanoscale objects.

TABLE 1
Electrical resistance and current noise of the submicron
pores with conical geometry that were used in this work.

| Diameter of pore | Resistance | Noise at 10 kHz^{a} | Noise at 1 kHz^{a} |
|---|---|---|---|
| (nm) | MW | (pA RMS) | (pA RMS) |
| 900 | 1.1 | 17.1 | 10.1 |
| 650 | 1.4 | 16.0 | 8.9 |
| 575 | 1.8 | 14.1 | 7.5 |

^{a}A digital Gaussian low-pass filter with the specified cutoff frequency was used. All noise values were obtained at an applied potential of 0.2 V in recording buffer (150 mM KCl; 50 mM tris(hydroxymethyl)aminomethane (TRIS), pH 7.8; 0.1 mg ml⁻¹ bovine serum albumin; 0.1% w/v Tween 20).

### EXAMPLE 4

Determination of the time resolution required for accurate extraction of quantitative information from Coulter counting analysis. Extracting quantitative data from Coulter counting experiments requires careful design of the recording system and the pore since these two entities determine the bandwidth of the measurement. The bandwidth is one of the most important aspects of the recorded data because it determines the time resolution. The time resolution of the measurement sets the upper bound of the “speed” at which changes in current can be recorded. That is, if a change in current occurs faster than the time resolution of the recording, then the recorded current “jumps” from one value to the next and the intervening information on how the current arrived at this value is lost. In the context of a Coulter counting experiment, the time resolution of the measurement determines the maximum resolution with which the resistive pulse of a particle can be observed while it passes though the pore. If it moves faster than the time resolution, then the peak amplitude of the resistive-pulse will be clipped. This clipping can cause inaccuracies in calculations that use the peak amplitude. Another important aspect of recording data accurately is the sampling frequency. According to the Nyquist theorem, the sampling frequency should always be at least 4 times the bandwidth of the recording.

In the absence of both filtering and series resistance compensation, the maximum possible bandwidth that is obtainable in a Coulter counting experiment is determined by the access resistance of the pore and the capacitance of the substrate. The resistance of the pore in parallel with the capacitance of the substrate that supports the pore forms a one-pole low-pass RC filter The maximum bandwidth of this filter can be estimated by the following equation:

$$B \leq \frac{1}{2 π RC} \qquad ( 8 )$$

where R is the resistance of the pore and C is the capacitance of the substrate. Since glass was used (a very good dielectric) and since the geometry of the pores used was conical (and did not include a thin, insulating membrane), the capacitance of the substrate was extremely low (<1 pF). Assuming a resistance of 1.8 MΩ (the maximum resistance of the pores fabricated), the theoretical bandwidth of the recording was 88 kHz. According to the manufacturer, the maximum bandwidth that was available from the recording system in the configuration that used was approximately 50 kHz (β=0.1, whole-cell mode); therefore the overall bandwidth of our measurement was not limited by the recording chip but by the amplifier. It was, however, considerably higher than the bandwidth of 8-9 kHz that was required for reliable analysis of the data recorded (see below).

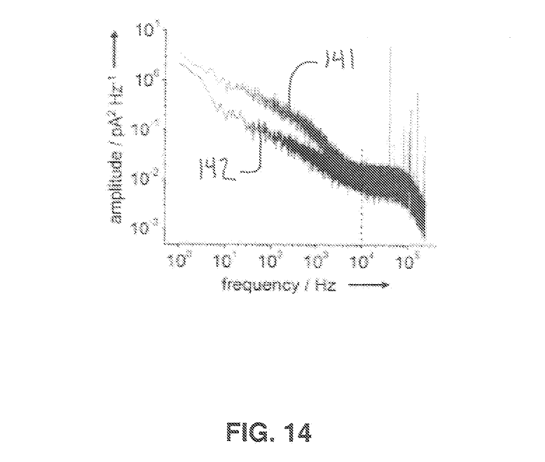

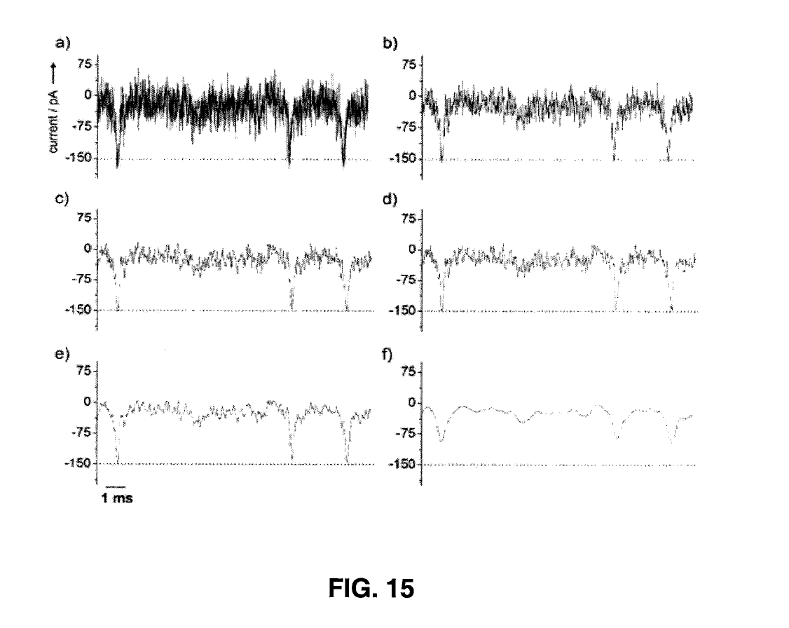

In order to determine the bandwidth required to measure accurately the amplitude of the current peaks, the power spectra of current traces were examined with and without events as shown in FIG. 14. These two power spectra show that an accurate detection of the events required a bandwidth of approximately 8-9 kHz. This result allowed reduction of the RMS noise of the current traces by filtering with a digital low-pass filter (Gaussian) with a cutoff frequency of 10 kHz. Since the events only required a bandwidth of 8-9 kHz, the amplitude of the peaks was not reduced by the 10 kHz filter as illustrated in FIG. 15c. In fact, FIG. 15e shows that filtering with a cutoff frequency as low as 5 kHz would have reduced the amplitude of the signal only marginally. Significant reduction in amplitude was observed when using a cutoff frequency of 1 kHz (FIG. 15f). After filtering all recorded data with a 10 kHz low-pass filter, the data were decimated to a sampling frequency of 50 kHz. As predicted by the Nyquist sampling theorem, this decimation also had no effect on the peak amplitude (FIG. 15c, d).

FIG. 14 shows the power spectra of original current traces with and without events (here immune complexes). Both current traces were recorded at maximum bandwidth of the recording setup (˜50 kHz). The power spectrum of the current trace with events 141 contained significantly more low frequency content than the power spectrum of the current trace without events 142. As determined from the plot, the maximum frequency component of the events was approximately 8-9 kHz. Therefore the current trace could be processed by low-pass filters with cutoff frequencies of 10 kHz (dotted line) without causing significant signal distortion. The average peak amplitude of the events in the current trace was 123±40 pA. The current traces were obtained from a pore with a diameter of 650 nm.

FIG. 15 shows the effect of the cutoff frequency used for low-pass filtering on the peak amplitudes of current events during passage of immune complexes through a submicron pore. The panels show: a) Current trace with three events after filtering with a digital (Gaussian) low-pass filter with a cutoff frequency of 50 kHz. b) Same current trace after filtering with a cutoff frequency of 20 kHz. The difference in the peak amplitude of the events between trace a) and b) was due to the reduction in current noise (from 26 to 17 pA RMS) and not due to clipping of the peak as a result of the reduced filter cutoff frequency. c) Same current trace after filtering with a cutoff frequency of 10 kHz. d) Same current trace as in c) but decimated to a sampling frequency of 50 kHz, instead of 500 kHz as in a)-c). As predicted by the Nyquist sampling theorem, the amplitude of the signal did not change significantly. e) Same current trace after filtering with a cutoff frequency of 5 kHz. The peak amplitude of the events decreased slightly since the cutoff frequency of the filter was below the maximum frequency component of the events (8-9 kHz, see FIG. 14). f) Same current trace after filtering with a cutoff frequency of 1 kHz. Since the cutoff frequency of the filter was significantly below the maximum frequency component of the events, the events are distorted and the peak amplitude has decreased by a factor of approximately 0.5. The digital filters were always applied to the original, high-bandwidth current trace. The resistive-pulses were caused by immune complexes passing through a pore with a diameter of 650 nm.

### EXAMPLE 5

Confirmation of formation of immune complexes by phase contrast and fluorescence microscopy. Immunoprecipitation experiments were performed in 0.5 mL vials in order to verify that the anti-mouse antibody formed immune complexes with the monoclonal antibody from mouse against baculovirus (here used as the antigen). The antibody and antigen were added to recording buffer at the concentrations listed in Table 2. The vials, each containing 20 μL of solution, were initially vortexed and then left at room temperature for ≧2 hours without agitation. The total volume was carefully removed from the vial, placed on a clean microscope slide, and covered with a clean cover glass.

TABLE 2
Antibody and antigen concentration used to verify
the formation of immune complexes by microscopy.

| | | | Total |
|---|---|---|---|
| | Polyclonal antibody | Antigen | protein |
| Vial | (μM) | (μM) | (μM) |
| 1 | 1.33 | 0 | 1.33 |
| 2 | 0 | 1.33 | 1.33 |
| 3 | 0.667 | 0.667 | 1.33 |

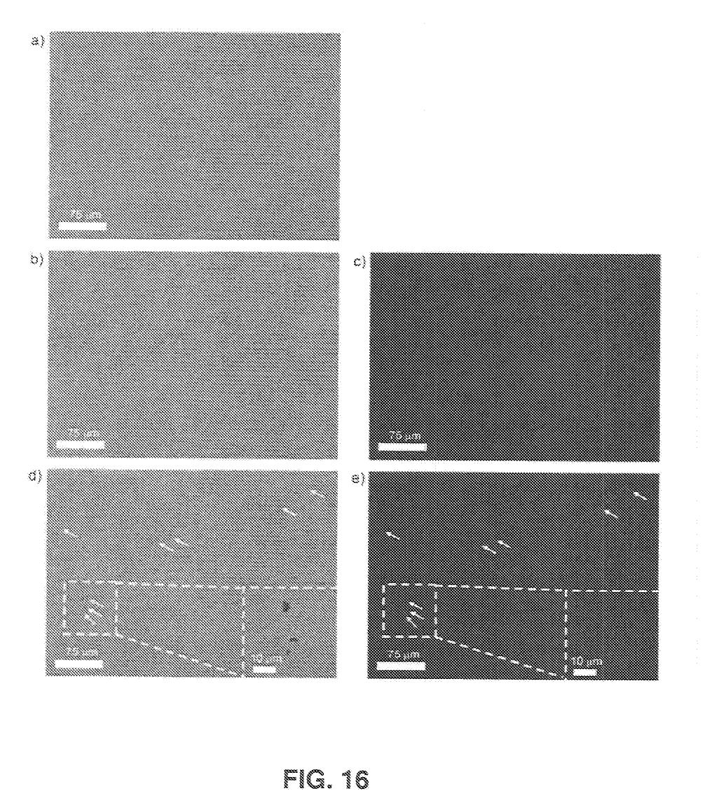

Slides were examined using a Nikon Eclipse TE 2000-U inverted microscope with a 20× objective in phase-contrast mode. No complexes were observed when only the antibody (FIGS. 16b, c) or the antigen (FIG. 16a) was present at a concentration of 1.33 μM. In contrast, when both the antigen and antibody were present at a concentration of 0.667 μM (total protein concentration=1.33 μM), immune complexes could be detected as shown in FIGS. 16d, e.

FIG. 16 shows microscope images to verify the specific formation of immune complexes. The panels show: a) Control experiment with the monoclonal antibody from mouse against baculovirus (antigen) at a concentration of 1.33 μM. No protein aggregates were seen on the slide by phase contrast microscopy. b) Control experiment with the anti-mouse antibody from goat that was labeled with tetramethylrhodamine isothiocyanate (TRITC) at a concentration of 1.33 μM. No protein aggregates were seen on the slide by phase contrast microscopy. c) False colored fluorescence image of the same field of view as in b). No protein aggregates of the fluorescently-labeled antibody were visible. d) Immunoprecipitation experiment with the antigen and anti-mouse antibody each at a concentration of 0.67 μM. The phase contrast image shows at least eight micron-sized immune complexes (indicated with white arrows). e) False colored fluorescence image of the same field of view as in d). A typical fluorescent filter set for rhodamine, an exposure time of 1 s, and the maximum intensity of excitation of the lamp (Exfo X-Cite 120, Photonic Solutions, Mississauga, Ontario) was used to capture this image. All of the images were captured with a CCD camera (Photometrics CoolSnap HQ, Roper Scientific, Trenton, N.J.) and processed using image analysis software (Metamorph, Universal Imaging, Downington, Pa.).

### EXAMPLE 6

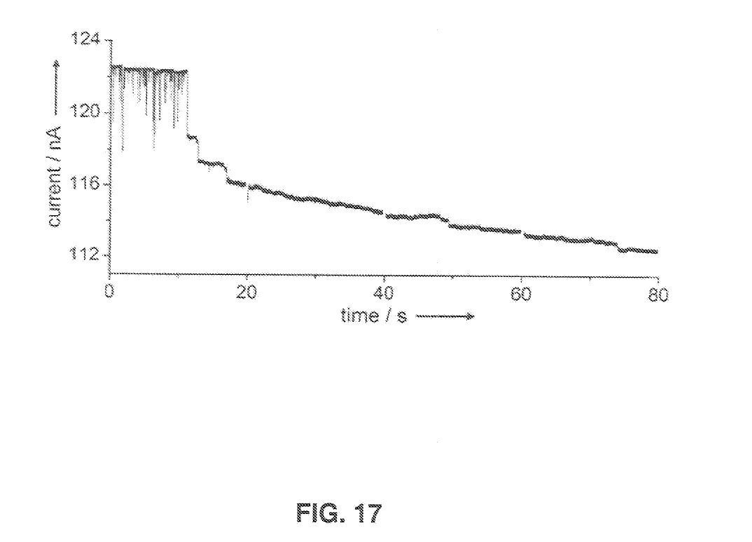

Blockage of submicron pores by biospecific formation of large immune complexes. As shown in FIG. 17, blockage of the submicron pore, with a diameter of 650 nm, by large immune complexes was detected. At a concentration of 151 nM monoclonal antibody from mouse against baculovirus (here used as the antigen) and 151 nM anti-mouse antibody, the resulting immune complexes grew large enough that they clogged the pore as indicated by step-wise increases in electrical resistance (blockage started approximately 15 minutes after addition of anti-mouse antibody). This “immunospecific blockage” may be useful for simple detection of antibody-antigen interactions. The graph is composed of several concatenated data files; a small gap separates each file. This method can of course be applied to nanopores as well.

### EXAMPLE 7

Sensing the formation of immune complexes in the presence of serum. FIG. 18 shows time courses of the formation of immune complexes in a solution containing serum. The panels show: a) Control experiment with 2 μL serum from a rabbit that was not immunized (filtered through a membrane with pores of 0.1 μm) added to 40 μL of recording buffer. Note the presence of small peaks that were caused by serum components not removed by the filter. Addition of the antigen (here mouse monoclonal antibody against baculovirus) to a final concentration of 151 nM did not cause any change in the signal. b) Anti-mouse antibody was dissolved in unfiltered rabbit serum and then this mixture was filtered using a membrane filter with 0.1 μm pores. A volume of 2 μL of rabbit serum containing anti-mouse antibody was added to 40 μL of recording buffer; the final concentration of antibody was 151 nM. Addition of the antigen to a final concentration of 355 nM caused a significant increase in the number of events and the size of the events. c) Antigen was added to a final concentration of 151 nM. As expected from FIG. 3a, no events resulted from passage of antigen alone through the pore. Addition of 2 μL of rabbit serum containing anti-mouse antibody to 42 μL of recording buffer initiated the formation of immune complexes; the final concentration of antibody was 151 nM. As seen in b), immune complexes rapidly formed causing a significant increase in the number of events and the size of the events. Each current recording is composed of multiple concatenated data files; a small gap separates each file. The time in minutes since addition of rabbit serum, or antigen is indicated above the beginning of each file. A pore with a diameter of 575 nm (FIG. 12a) was used for all experiments.

### EXAMPLE 8

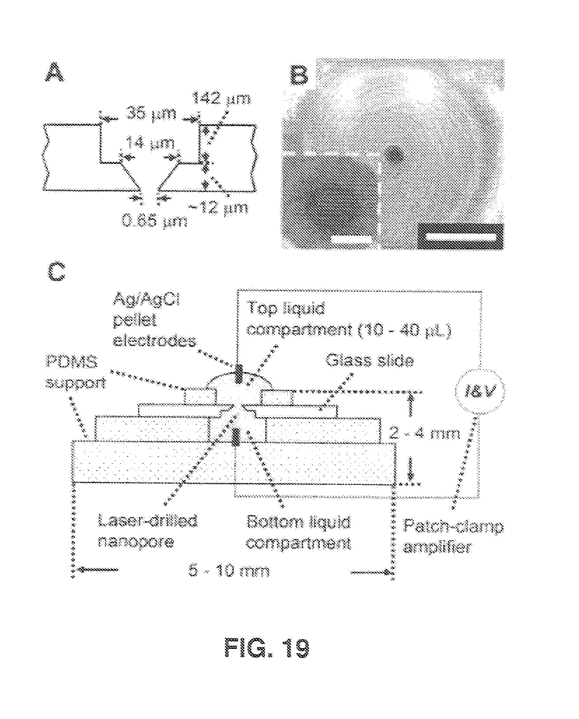

Recording setup. FIG. 19 shows a schematic design of the conical pore and the recording setup. The panels show: (A) Geometry and dimensions of the pore used in all experiments. (B) Scanning electron microscope (SEM) image looking into the 35 μm cylinder of the pore shown in A; scale bar=5 μm. The inset shows a close-up of the narrowest part of the pore; scale bar=500 nm. (C) Sideview of the experimental setup. A patch-clamp amplifier applied a constant voltage and detected small changes in current (pA-range) with a bandwidth of ˜50 kHz (the sampling frequency was 500 kHz). A poly(dimethylsiloxane) (PDMS) fluidic setup allowed for replacement of solution on either side of the pore. The electrode in the top liquid compartment was polarized positively (+0.2 V) and virus particles were added to this compartment.

Solutions: All solutions were prepared with deionized water (resistivity of 18.2 MΩcm, Aqua Solutions, Jasper, Ga.) and potassium chloride, sulfuric acid (both from EMD Biosciences, La Jolla, Calif.), tris(hydroxymethyl)aminomethane (TRIS; Shelton Scientific, Shelton, Conn.), bovine serum albumin (Sigma, St. Louis, Mo.), Tween 20 (Mallinckrodt Chemicals, Phillipsburg, N.J.), hydrochloric acid (VWR International, West Chester, Pa.), nitric acid (Fluka Chemie, Buchs, Switzerland), and hydrogen peroxide (EMD Chemicals, Gibbstown, N.J.) were used without further purification. Recording buffer, composed of 150 mM KCl, 50 mM TRIS buffer, pH 7.8, 0.1 mg mL⁻¹ bovine serum albumin, 0.1% w/v Tween 20, was filtered through sterile, low-protein-absorption polyethersulfone membrane filters with a pore size of 0.2 μm (Pall, East Hills, N.Y.). Concentrated PBCV-1 virions and the polyclonal antiserum from rabbit were both kindly provided by J. L. Van Etten (University of Nebraska-Lincoln). The virus and antiserum were diluted in recording buffer and the antiserum solution was filtered through a 0.2-μm membrane filter.

Mixing and data analysis: The diluted virus solution was added to the buffer in the top liquid compartment (final volume of this mixture was 40 μL). To keep the concentration of the polyclonal antibodies constant, 2 μL of the diluted antiserum was always added to this virus/buffer mixture. The volume in the top liquid compartment was then aspirated and expelled three times using a pipette (Eppendorf Reference, Westbury, N.Y.) with a volume setting of 5 μL. This procedure combined with the small volume ensured that the two solutions were well mixed.

The addition of the virus to the top liquid compartment caused the RMS noise (filter cutoff frequency=10 kHz) to change by a maximum of 15.5% (15.6-18.0 pA RMS at a virus concentration of 4.4×10⁸ particles mL⁻¹). This change was, however, not correlated with the concentration of the virus; the maximum concentration of virus (4×10⁹ particles mL⁻¹) caused a change of only 3%. Addition of the antiserum to the top liquid compartment caused the RMS noise to change by less than 4%.

During the data analysis, immediately after the addition of antiserum or control serum the peak amplitude from virus particles of the events was slightly reduced (<4.7%). This decrease in amplitude was attributed to a small change in the conductance of the solution. In order to minimize the error in our determination of the number of antibodies bound to a virus, the average of the Gaussian means of the peak amplitudes was used, which was measured immediately after addition of antiserum (before significant binding of antibodies could occur) as the peak amplitude of virus particles that did not have antibodies bound on their surface.

Data acquisition and processing: Prior to each experiment, the glass cover slide that contained the pore was cleaned in a fresh mixture of 3:1 concentrated sulfuric acid to 30% hydrogen peroxide for at least 15 min. The poly(dimethylsiloxane) (PDMS, Sylgard 184 Silicone, Dow Corning, Midland, Mich.) support that contained the bottom liquid compartment was cleaned thoroughly after each experiment with alternating rinses of deionized water and 95% ethanol (VWR International). The PDMS film that was used for the top liquid compartment was cut from a slab of PDMS that was cured in a clean Petri dish; a new PDMS film was used in each experiment. This procedure ensured a good seal between the PDMS and the glass, and no leaks were encountered during the experiments. Ag/AgCl pellet electrodes (Eastern Scientific, Rockville, Md.) were used since the recorded currents were relatively large (≈140 nA). A patch-clamp amplifier (Axopatch 200B) was used in voltage clamp mode and the analog low-pass filter was set to a cutoff frequency of 100 kHz. The setup was completed by a low-noise digitizer (Digidata 1322, sampling frequency set to 500 kHz), and a computer with recording software (Clampex 9.2) for data acquisition. For all data processing and event collection, Clampfit 9.2 (all from Axon Instruments, Union City, Calif.) was used.

Data was filtered with a digital Gaussian low-pass filter with a cutoff frequency of 10 kHz and then decimated to a sampling frequency of 50 kHz (see Example 10 for detailed analysis of the bandwidth of the measurement, the bandwidth and sampling frequency required to resolve an event due to a virus completely, and the effects of digital filtering and decimation of data on the peak amplitudes and half-widths of the events). A peak was defined as an “event” due to passage of a virus if the signal had an amplitude of at least 13 times the standard deviation of the baseline signal from its mean for a duration of at least 25 μs and a maximum of 10 ms (all events had a halfwidth >100 μs). The collected data was analyzed using Origin 7.5 (OriginLab, Northampton, Mass.) and Matlab (The MathWorks, Natick, Mass.).

### EXAMPLE 9

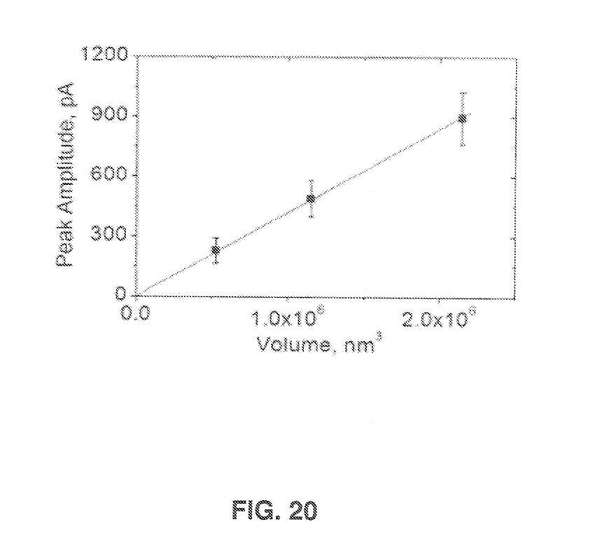

Peak amplitude versus particle volume in conical submicrometer pores. FIG. 20 graphically depicts a plot of the average peak amplitude of the resistive-pulses caused by particles with a diameter of 100, 130, and 160 nm passing through a pore with a diameter of 575 nm versus particle volume. The data were fitted using a linear regression algorithm that required the line to pass through the origin; the slope of the line was 4.2×10⁻⁴ pA nm⁻³. A slope of 3.9×10⁻⁴ pA nm⁻³ was obtained for the pore with a diameter of 650 nm (FIGS. 19A, B).

### EXAMPLE 10

Determination of the bandwidth required for accurate extraction of quantitative information from Coulter counting analysis. Extracting quantitative data from Coulter counting experiments requires careful design of the recording system and the pore since these two entities determine the bandwidth of the measurement. The bandwidth is one of the most important aspects of the recorded data because it determines the time resolution. The time resolution of the measurement sets the upper bound of the “speed” at which changes in current can be recorded. That is, if a change in current occurs faster than the time resolution of the recording, then the recorded current “jumps” from one value to the next and the intervening information on how the current arrived at this value is lost. In the context of a Coulter counting experiment, the time resolution of the measurement determines the maximum resolution with which the resistive pulse of a particle can be observed while it passes though the pore. If the particle moves faster than the time resolution, then the peak amplitude of the resistive-pulse will be clipped. This clipping can cause inaccuracies in calculations that are based on the peak amplitude. Another important aspect of recording data accurately is the sampling frequency. According to the Nyquist theorem, the minimum sampling frequency required to prevent aliasing is twice the signal bandwidth (i.e., if the signal has a bandwidth of 10 kHz, the sampling frequency must be at least 20 kHz); however, it is typically recommended that a sampling rate at least 5 times the signal bandwidth be used.

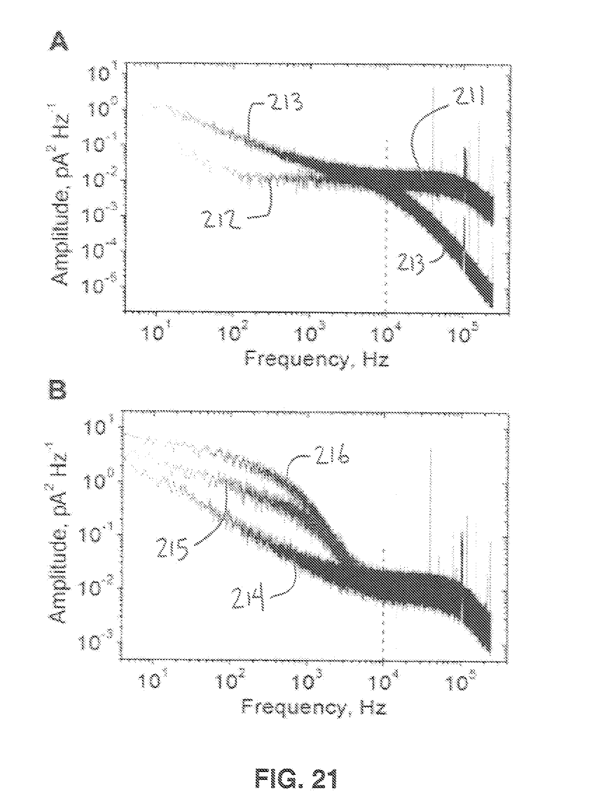

The maximum possible bandwidth that is obtainable in a Coulter counting experiment is determined by the geometry of the pore, the substrate material, the conductivity of the buffer, and the recording electronics. In order to determine the bandwidth that was available during our experiments, the power spectrum of a high bandwidth current trace (taken at the maximum bandwidth of the recording setup, 4-pole Bessel filter with cutoff frequency of 100 kHz and a sampling rate of 500 kHz; see power spectrum 211 in FIG. 21A) was examined. The power spectrum contained a linear decrease in power between 4-1000 Hz and a roll-off in power after ˜50 kHz. The linear drop in the range of 4-1000 Hz is most likely due to 1/f noise. This hypothesis is supported by the reduction in power (noise) seen in this frequency range when the applied voltage was decreased to 0 V as shown by the power spectrum 212 in FIG. 21A. Therefore the decrease in power in the range from 4-1000 Hz is not due to a limited bandwidth of the recording setup or pore but rather to the reduction of 1/f noise with increasing frequency.

FIG. 21 illustrates the determination of the bandwidth available during Coulter counting experiments and the bandwidth required to resolve events. The panels show: (A) Power spectra of current traces under three conditions: (211)—no digital filtering and an applied voltage of 0.2 V, (212)—no digital filtering and an applied voltage of 0 V, and (213)—same current trace used as in plot 211 after digitally filtering with a 1-pole RC filter with a cutoff frequency of 10 kHz. Based on these power spectra, the bandwidth of the Coulter counting apparatus (patch clamp amplifier and submicrometer pore) was ˜50 kHz. (B) Power spectra of high bandwidth traces (˜50 kHz; no digital filtering) without events from viruses (214), with events from viruses (215), and with events 12 minutes after addition of antiserum (216). As illustrated by this plot, the maximum frequency component of the virus events was ≦8 kHz. The concentration of the virus was 2.8×10⁸ virus particles mL⁻¹ and the antiserum was added to the top liquid compartment such that the final dilution was 0.001× the original antiserum.

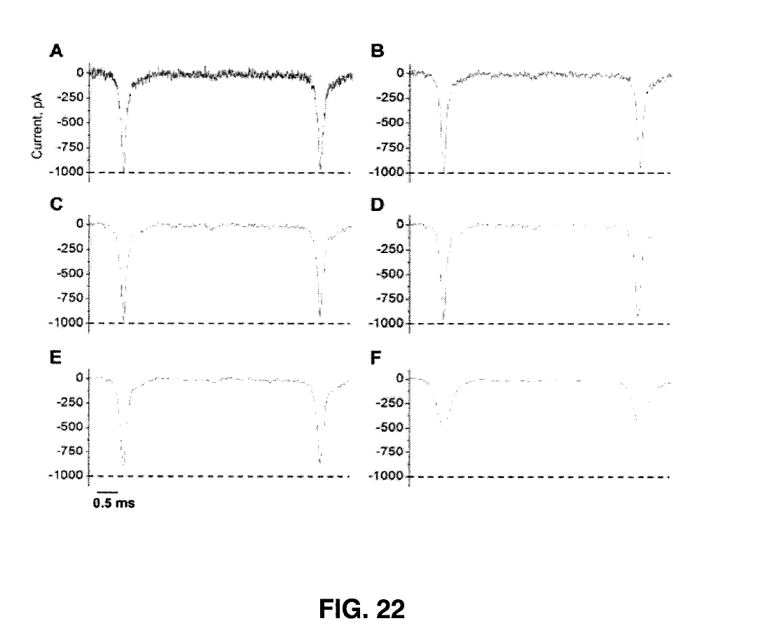

The parameter that is important here, namely the reduction of bandwidth due to the recording setup, can be obtained from the “roll-off” at higher frequencies. This roll-off begins at ˜50 kHz and is most likely due to a combination of two factors: the bandwidth limitation of the headstage (according to Axon Instruments, the headstage operating in the configuration used, i.e., whole cell mode with β=0.1, has a bandwidth of ˜50 kHz) and the 4-pole Bessel filter that was used to prevent aliasing (cutoff frequency of 100 kHz). The analysis of the power spectra in FIG. 22 therefore shows that the available bandwidth was ˜50 kHz.

Since the pore can be modeled as a network of resistive and capacitive components, it is possible that the pore itself could act as a filter. Due to the geometry of the pore, the model circuit is complicated, and this result makes a direct derivation of the filtering characteristics (i.e., the transfer function) difficult. If the pore would constitute a significant filter, then it can be expected that the pore would act as a single pole (or multi pole) RC filter. In order to illustrate the hypothetical effect of such a filter, the original current trace was filtered with a single pole RC filter with a cutoff frequency of 10 kHz (arbitrarily chosen) and the power spectrum was recalculated, which is shown in FIG. 21A as trace 213. As expected, even this simple one pole filter causes a significant change in the power spectrum of the plot. Therefore, in the frequency range of interest in this work, the submicrometer pore structure did not appear to be acting as a filter and the bandwidth of the measurement was not limited by the pore but rather by the recording electronics to ˜50 kHz (it would be conceivable that the pore was acting like a filter with a cutoff frequency close to 50 kHz, which would overlap with the roll-off of the amplifier electronics; however, calculations based on the resistive and capacitive components of the pore suggest a cutoff frequency >500 kHz).

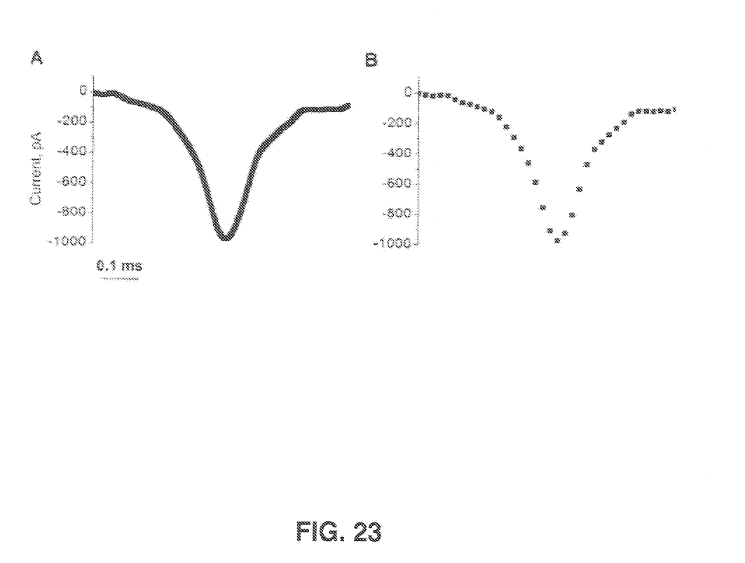

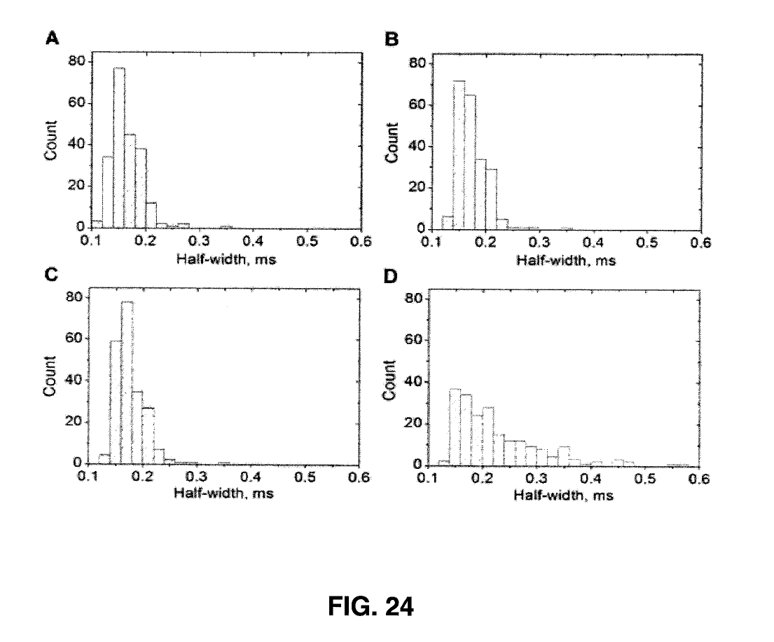

Power spectrum analysis was also used to determine the bandwidth required to resolve events due to viruses with or without antibody bound. As shown in FIG. 21B, the current traces that contained events had more power in frequencies ranging from 4-8000 Hz compared to the trace that did not contain events. Therefore, a bandwidth of ˜8 kHz was required to resolve the events completely. Due to this result, the RMS noise of the current traces was reduced by filtering with a digital low-pass filter (Gaussian) with a cutoff frequency of 10 kHz without causing significant distortion of events: as expected, the amplitude of the virus peaks was not significantly reduced (<5% decrease) by the 10 kHz filter when compared to the peaks that were filtered at 50 kHz as illustrated in FIGS. 22A, C (more than 200 events were also examined and their mean peak amplitude and the mean value of a Gaussian curve fit to the peak amplitude distribution decreased by less than 5%). Similarly, the mean half-width value of over 200 events changed by less than 8% due to the 10 kHz filter (FIGS. 24A, B). FIG. 22E demonstrates that filtering with a cutoff frequency as low as 5 kHz would have only reduced the peak amplitude of the signal by less than 11%. Significant reduction in amplitude would have been observed, however, if a cutoff frequency of 1 kHz was used as shown in FIG. 22F (˜50% decrease). In the work presented here, the recorded data were filtered with a 10 kHz low-pass filter, and decimated to a sampling frequency of 50 kHz. As predicted by the Nyquist sampling theorem, this decimation had a minimal effect on the peak amplitude (FIGS. 22C, D and FIG. 23 show that the decimation of data caused a negligible change in the peak amplitude of an event; the mean peak amplitude and the mean value of a Gaussian fit of over 200 events decreased by less than 1%). Decimation also had a negligible effect on the event half-width (FIG. 23 and FIGS. 24B, C show that the decimation of data caused the mean half-width of over 200 events to decrease by less than 1%).

FIG. 22 shows the effect of the cutoff frequency used for low-pass filtering on the peak amplitudes of current events during passage of viruses through a submicron pore. The panels show: (A) Current trace with two events after filtering with a digital (Gaussian) low-pass filter with a cutoff frequency of 50 kHz. (B) Same current trace after filtering with a cutoff frequency of 20 kHz. (C) Same current trace after filtering with a cutoff frequency of 10 kHz. (D) Same current trace as in C but decimated to a sampling frequency of 50 kHz, instead of 500 kHz as in A-C. As predicted by the Nyquist sampling theorem, the amplitude of the signal did not change significantly (see FIG. 23). (E) Same current trace as in A-C after filtering with a cutoff frequency of 5 kHz. Under these conditions, the peak amplitude of the events decreased slightly since the cutoff frequency of the filter was below the maximum frequency component of the events (˜8 kHz, see FIG. 21). (F) Same current trace after filtering with a cutoff frequency of 1 kHz. Since the cutoff frequency of the filter was significantly below the maximum frequency component of the events, the events are distorted and the peak amplitude has decreased by a factor of approximately 0.5. The digital filters were always applied to the original, high-bandwidth current trace.

FIG. 23 illustrates a close-up view of a single event due to the passage of a virus through the pore before and after decimation of data. The panels show: (A) Close-up view of a single event after filtering with a digital low-pass filter with a cutoff frequency of 10 kHz (sampling frequency of 500 kHz). (B) Same trace as in A decimated by a factor of ten (sampling frequency of 50 kHz). The change between the peak amplitude and half-width of trace A and trace B was smaller than 1%.

FIG. 24 graphically depicts histograms of the half-widths of events due to the passage of viruses at different bandwidths in the absence and presence of antiserum. The graphs demonstrate that the bandwidth and data decimation used did not distort the recorded signals (i.e., was sufficient to resolve the entire signal). The panels show: (A) Half-widths of events due to the passage of viruses after filtering with a digital Gaussian low-pass filter with a cutoff frequency of 50 kHz. (B) Same events as in A but filtered with a low-pass filter with a cutoff frequency of 10 kHz. (C) Same events as in B but after decimation to a sampling frequency of 50 kHz. Out of all virus events collected (after digital filtering and decimation), less than 3% of events had a half-width less than 0.12 ms, and all events had a half-width greater than 0.10 ms. (D). Half-widths of events collected 10.5-14.5 minutes after addition of antiserum (digital filter cutoff of 50 kHz, sampling frequency decimated to 50 kHz). The concentration of the virus was 2.8×10⁸ virus particles×mL⁻¹ and the antiserum was added to the top liquid compartment such that the final dilution was 0.001× the original antiserum.

### EXAMPLE 11

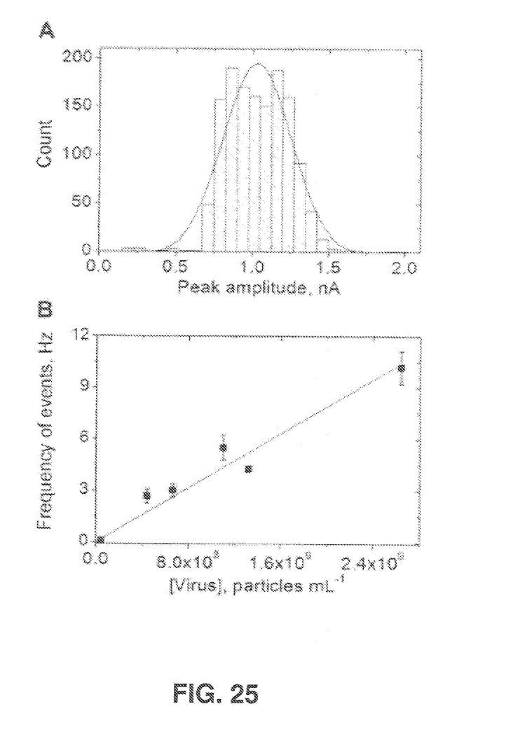

Analysis of the measured diameter of PBCV-1 and frequency of events versus virus concentration. The measured diameter of PBCV-1 (203±14 nm) had a standard deviation (STD) of ˜7%. Previous reports in the literature on using resistive-pulse sensing to size virus particles have resulted in STDs of ≦4%. The STD of ˜7% reported here may be due to one of following three effects, or to a combination of these effects. First, the data used to create the histogram in FIG. 25A was collected from 5 separate experiments that were conducted over seven days. Although the procedure for the experiments was always the same, there may have been small differences (e.g., in temperature or recording buffer) that caused an increase in the STD. Second, virus particles may have passed through the pore off center which could lead to off-axis effects that can increase the STD of a population of particles by as much as 3.5%. Finally, while unlikely, the STD of ˜7% could be due the existence of structural variants of PBCV-1 (i.e., the population of PBCV-1 particles may have multiple distinct diameters).

FIG. 25 graphically depicts: (A) Histogram of the peak amplitudes of 1395 events caused by PBCV-1 without antibody bound passing through the pore shown in FIGS. 19A, B. The histogram was fit with a Gaussian distribution. (B) Frequency of events versus the concentration of virus. The data points were fit using a linear regression algorithm that required the line to pass through the origin; the slope of the line was 4.0×10⁻⁹ Hz×mL×virus particles⁻¹.

### EXAMPLE 12

Concentration of antibodies specific for PBCV-1 in the rabbit antiserum. The concentration of specific antibody in the rabbit antiserum was unknown. However, a lower bound was obtained for the concentration of the specific antibody in the antiserum based on the number of antibodies bound per virus at equilibrium and the concentration of the virus. At the highest concentration of virus (4×10⁹ particles×mL⁻¹), approximately 550 antibodies were bound to each virus at equilibrium. Therefore there were at least 4×10⁹×550=2.2×10¹² specific antibodies×mL⁻¹ present in the diluted serum. Based on the molecular weight of an IgG antibody of 150,000 Daltons, this value corresponds to a specific antibody concentration of 5.5×10⁻⁴ mg×mL⁻¹. Since the serum was diluted by a factor of 1000, the original serum contained at least 0.55 mg×mL⁻¹ of specific and active antibody. This lower bound compares favorably to a previous study that reported an average concentration of specific antibody of 0.78 mg×mL⁻¹ in rabbit antiserum.

### EXAMPLE 13

Pore blockage by aggregates of virus. In the experiments that involved antiserum (the antiserum dilution was held constant at 0.001× the original antiserum, the virus concentration was varied), the pore eventually blocked (>8 min after addition of antiserum) due to the formation of large viral aggregates. No more events could be recorded after blockage (at the lowest virus concentration only partial blockage occurred). This blockage terminated the experiment, and before the next use, the pore was cleaned in a fresh mixture of 3:1 concentrated sulfuric acid to 30% hydrogen peroxide.

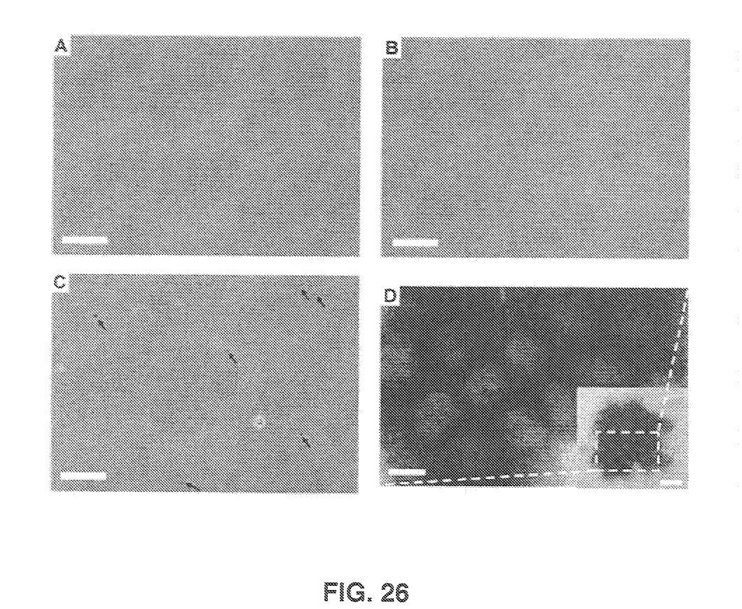

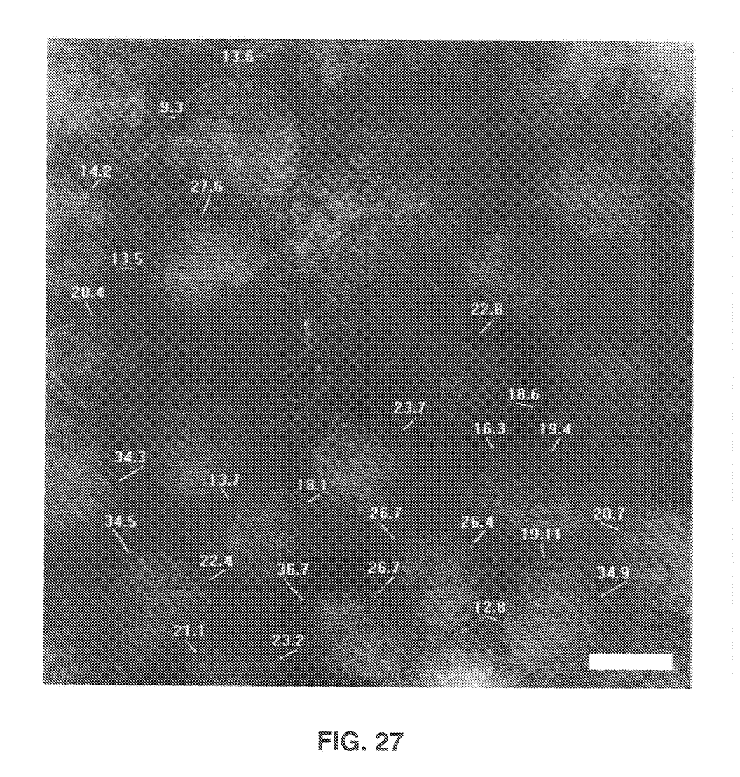

FIG. 26 shows microscopic observation of antiserum, control serum, and of virus antibody complexes. The panels show: (A) Phase contrast microscope image of the antiserum at a dilution of 0.001 in the absence of virus particles; scale bar=75 μm. (B) Phase contrast microscope image of control serum at a dilution of 0.001 in the presence of virus at a concentration of 6×10⁸ virus particles×mL⁻¹; scale bar=75 μm. (C) Phase contrast microscope image of immune complexes formed by the antiserum at a dilution of 0.001 and the virus at a concentration of 6×10⁸ virus particles×mL⁻¹. The black arrows indicate micrometer-sized viral aggregates; scale bar=75 μm. (D) Transmission electron microcopy (TEM) image of virus aggregated by antibody. The average distance between viruses in the aggregate was 23±7 nm which is close to the maximum span (˜15 nm) of an IgG molecule (see FIG. 27). The serum was used at a dilution of 0.001 and PBCV-1 was used at a concentration of 1×10⁹ particles×mL⁻¹. Scale bar=100 nm. The inset shows the entire aggregate. Scale bar of the inset=300 nm. The buffer used for all images was composed of 150 mM KCl, 50 mM tris(hydroxymethyl)aminomethane (TRIS) buffer, pH 7.8.

FIG. 27 shows a TEM image with individual measurements of the distance between virus particles in an aggregate. The serum was used at a dilution of 0.001 and PBCV-1 was used at a concentration of 1×10⁹ particles×mL⁻¹. All of the measurements are in nm. Scale bar=100 nm.

Preparation of virus samples for TEM. A 300 mesh copper carbon grid (Electron Microscopy Sciences, Hatfield, Pa.) was placed in a glow discharge for 1 minute at 100 millitorr and 60 volts (Denton Vacuum DV-502, Moorestown, N.J.) to increase the hydrophilicity of the grid. The serum was diluted 1000 fold and incubated with the virus at a concentration of 1×10⁹ particles×mL⁻¹ in buffer containing 150 mM KCl, 50 mM tris(hydroxymethyl)aminomethane (TRIS) buffer, pH 7.8 for 1 hour. A drop of this solution containing the virus and antiserum was placed on the hydrophilic grid and the solution was wicked away with a kimwipe paper (Kimberly-Clark, Neenah, Wis.). For negative staining of the antibody-virus aggregates, a drop of 1% phosphotungstic acid was placed on the grid for ˜2 minutes and the solution was then wicked away with a kimwipe. The aggregates were imaged using a transmission electron microscope (Phillips CM100, FEI Company, Hillsboro, Oreg.) at 60 kV.

The examples and other embodiments described herein are exemplary and not intended to be limiting in describing the full scope of compositions and methods of this technology. Equivalent changes, modifications and variations of specific embodiments, materials, compositions and methods may be made within the scope of the present technology, with substantially similar results.

What is claimed is:

1. A method for detecting assembly of complexes, the complexes formed of submicrometer objects comprising:
providing a solution where a first portion is separated from a second portion via a submicrometer pore;
adding a submicrometer object to the first portion of the solution, wherein the submicrometer object associates with another submicrometer object to produce a complex; and
detecting passage of the complex from the first portion of the solution through the submicrometer pore to the second portion of the solution using resistive pulse sensing.

2. The method for detecting the assembly of complexes according to claim 1, wherein the detecting passage of the complex from the first portion of the solution through the submicrometer pore to the second portion of the solution using resistive pulse sensing includes a complex comprising at least two submicrometer objects.

3. The method for detecting the assembly of complexes according to claim 1, wherein the detecting passage of the complex from the first portion of the solution through the submicrometer pore to the second portion of the solution using resistive pulse sensing includes detecting a change in current, the change in current being proportional to the volume of the complex.

4. The method for detecting the assembly of complexes according to claim 1, wherein the detecting passage of the complex from the first portion of the solution through the submicrometer pore to the second portion of the solution using resistive pulse sensing includes detecting a change in current, the change in current being proportional to the volume of the complex as determined by a surface that surrounds the objects the in the complex, the total volume of the objects in the complex if the complex is porous, or not proportional to either volume.

5. The method for detecting the assembly of complexes according to claim 1, wherein the detecting passage of the complex from the first portion of the solution through the submicrometer pore to the second portion of the solution using resistive pulse sensing includes detecting a number of resistive-pulses per time interval, the number of resistive-pulses per time interval being representative of the concentration of the complex.

6. The method for detecting the assembly of complexes according to claim 1, wherein the detecting passage of the complex from the first portion of the solution through the submicrometer pore to the second portion of the solution using resistive pulse sensing includes detecting a residence time of the complex in the submicrometer pore, the residence time being representative of the velocity of the complex.

7. The method for detecting the assembly of complexes according to claim 1, wherein the detecting passage of the complex from the first portion of the solution through the submicrometer pore to the second portion of the solution using resistive pulse sensing further includes detecting blockage of the submicrometer pore by the complex.

8. The method for detecting the assembly of complexes according to claim 1, wherein the adding a submicrometer object to the first portion of the solution includes a submicrometer object comprising a preassembled complex.

9. The method for detecting the assembly of complexes according to claim 1, wherein the detecting passage of the complex further comprises:
detecting passage of a first complex; and
detecting passage of a second complex, the second complex having a different number of submicrometer objects than the first complex.

10. The method for detecting the assembly of complexes according to claim 1, wherein the adding a submicrometer object to the first portion of the solution further comprises:
adding a first submicrometer object; and
adding a second submicrometer object, the second submicrometer object being different from the first submicrometer object.

11. The method for detecting the assembly of complexes according to claim 9, wherein the detecting passage of the complex from the first portion of the solution through the submicrometer pore to the second portion of the solution using resistive pulse sensing includes a complex comprising the first submicrometer object and the second submicrometer object.

12. The method for detecting the assembly of complexes according to claim 10, further comprising:
quantifying the number of the first submicrometer objects relative to the number of the second submicrometer objects in the complex.

13. The method for detecting the assembly of complexes according to claim 9, wherein the first submicrometer object is a monoclonal antibody; a polyclonal antibody; a submicrometer particle; a nanoparticle; a submicrometer particle or a nanoparticle with at least one immobilized functional group, protein, or polynucleotide; or a polynucleotide; and the second submicrometer object is a bacterial antigen; a mammalian antigen; a viral antigen; a viral particle; a membrane fragment containing an antigen or multiple antigens; a submicrometer particle; a nanoparticle; a submicrometer particle or a nanoparticle with at least one immobilized antigen, functional group, protein, or polynucleotide; or a polynucleotide.

14. A method for identifying intermolecular interactions comprising:
partitioning an electrolyte volume with a submicrometer pore;
providing a complex including a first submicrometer object and a second submicrometer object;
establishing a concentration gradient of the complex across the submicrometer pore; and
measuring a change in electrical signal when the complex traverses the submicrometer pore.

15. The method for identifying intermolecular interactions according to claim 14, wherein the providing a complex including a first submicrometer object and a second submicrometer object includes first and second submicrometer objects that are label-free.

16. The method for identifying intermolecular interactions according to claim 14, wherein the measuring a change in electrical signal when the complex traverses the submicrometer pore further comprises estimating the solid phase affinity constant.

17. The method for identifying intermolecular interactions according to claim 14, wherein the measuring a change in electrical signal when the complex traverses the submicrometer pore further comprises determining the volume of the complex based on the change in current.

18. The method for identifying intermolecular interactions according to claim 14, wherein the measuring a change in electrical signal when the complex traverses the submicrometer pore further comprises determining the concentration of complex based on the number of resistive pulses per time interval.

19. The method for identifying intermolecular interactions according to claim 14, wherein the measuring a change in electrical signal when the complex traverses the submicrometer pore further comprises determining the velocity of the complex based on the residence time of the complex in the submicrometer pore.

20. The method for identifying intermolecular interactions according to claim 14, wherein the providing a complex including a first submicrometer object and a second submicrometer object comprises a first submicrometer object and a second submicrometer object that are identical.

21. The method for identifying intermolecular interactions according to claim 14, wherein the providing a complex including a first submicrometer object and a second submicrometer object comprises a first submicrometer object and a second submicrometer object that are different.

22. The method for identifying intermolecular interactions according to claim 21, wherein the second submicrometer object can bind more than one first submicrometer object.

23. The method for identifying intermolecular interactions according to claim 22, wherein the measuring a change in electrical signal when the complex traverses the submicrometer pore further comprises:
estimating the number of the first submicrometer objects in a complex comprising at least one second submicrometer object and at least two first submicrometer objects.

24. The method for identifying intermolecular interactions according to claim 22, wherein the first submicrometer object is an antibody and the second submicrometer object is a viral particle.
